(19) United States
(12) Patent Application Publication
(10) Pub. No.: US 2025/0368999 A1
(43) Pub. Date: Dec. 4, 2025

(54) SOLUBLE INTERLEUKIN-7 RECEPTOR (SIL7R) MODULATING THERAPY TO TREAT CANCER

(71) Applicants: Mariano A. GARCIA-BLANCO, Galveston, TX (US); Gaddiel GALARZA-MUNOZ, Galveston, TX (US)

(72) Inventors: Mariano A. GARCIA-BLANCO, Galveston, TX (US); Gaddiel GALARZA-MUNOZ, Galveston, TX (US)

(73) Assignee: Board of Regents, The University of Texas System, Austin, TX (US)

(21) Appl. No.: 18/251,235

(22) PCT Filed: Oct. 29, 2021
(86) PCT No.: PCT/US2021/057419

§ 371 Date: Apr. 29, 2023

Related U.S. Application Data
(60) Provisional application No. 63/107,382, filed on Oct. 29, 2020.

(51) Int. Cl. C12N 15/113; A61K 45/06; A61P 35/00

(52) U.S. Cl. CPC C12N 15/1138; A61K 45/06; A61P 35/00; C12N 2310/11; C12N 2320/33

(57) ABSTRACT

The present invention includes compositions and methods for treating an autoimmune disorder or a cancer in a subject in need thereof, the method comprising: administering an effective amount of a composition comprising an oligonucleotide that specifically binds a complementary sequence of the Interleukin-7 receptor (IL7R) pre-mRNA that influences splicing of exon 6, wherein the SM-ASO decreases or increases exclusion of exon 6 in IL7R pre-mRNAs and respectively decreases or increases expression of the soluble isoform of IL7R (sIL7R). In certain embodiments, the oligonucleotide is an antisense oligonucleotide (ASO), or a splice-modulating antisense oligonucleotide (SM-ASO).

### STATEMENT OF FEDERALLY FUNDED RESEARCH

This invention was made with government support under F32-NS087899 awarded by NIH. The government has certain rights in the invention.

### TECHNICAL FIELD OF THE INVENTION

The present invention relates in general to the field of novel therapies that reduce or increase soluble IL7R (sIL7R) to treat autoimmune diseases (e.g., multiple sclerosis) or cancer, respectively.

### BACKGROUND OF THE INVENTION

The invention is based on discoveries of an autoimmunity pathway driven by elevated expression of sIL7R, and thus without limiting the scope of the invention, its background is described in connection with multiple sclerosis, as an example.

Multiple Sclerosis (MS) is a chronic autoimmune disease characterized by self-reactive immune cell-mediated damage to neuronal myelin sheaths in the central nervous system (CNS) that leads to axonal demyelination, neuronal death and progressive neurological dysfunction. Up to date, there is no cure for the disease and available treatments can only slow down disease progression, often by globally suppressing the immune system, causing a plethora of adverse side effects that could be severe or lethal. This global immunosuppression is the major limitation of current therapies.

The breach of immunological tolerance that leads to MS is thought to originate from complex interactions between environmental and genetic factors. Under this view, the genetic background of an individual could generate an environment permissive for the survival of self-reactive lymphocytes, which could be subsequently activated by the presence of an environmental trigger, usually in the form of an infectious agent.

The present inventors and others have previously shown that the variant rs6897932 (C/T, where C is the risk allele) within exon 6 of the Interleukin-7 receptor (IL7R) gene is strongly associated with increased MS risk (Gregory et al., 2007; International Multiple Sclerosis Genetics et al., 2007; Lundmark et al., 2007). Furthermore, the present inventors showed that the risk ‘C’ allele of this variant increases skipping of the exon (Evsyukova et al., 2013; Gregory et al., 2007), leading to up-regulation of sIL7R (Hoe et al., 2010; Lundstrom et al., 2013). Importantly, sIL7R has been shown to exacerbate the clinical progression and severity of the disease in the Experimental Autoimmune Encephalomyelitis (EAE) mouse model of MS, presumably by potentiating the bioavailability and/or bioactivity of IL7 cytokine (Lundstrom et al., 2013). This potentiation of IL7 by sIL7R enhances homeostatic expansion of both CD4⁺ and CD8⁺ T cells (Lundstrom et al., 2013). Further supporting a role of sIL7R in the pathogenesis of multiple sclerosis, and perhaps autoimmunity in general, elevated levels of sIL7R protein or RNA have been reported in patients of multiple sclerosis (McKay et al. 2008), rheumatoid arthritis (Badot et al., 2011), type 1 diabetes (Monti et al., 2013), and systemic lupus erythematosus (Lauwerys et al., 2014), wherein the levels of sIL7R correlate with disease activity. Collectively, these data link elevated levels of sIL7R to the pathogenesis of MS and autoimmunity, and position alternative splicing of IL7R exon 6 as a novel therapeutic target for MS and autoimmunity.

These references teach that sIL7R increases expansion of T cells leading to enhanced self-reactive responses, like those needed to kill cancer cells. Accordingly, up-regulation of sIL7R can be used as a novel immunotherapy to fight cancer, whether as a monotherapy or a combinatorial therapy with other anti-cancer agents, and a need remains for novel composition and methods for targeting the up-regulation of sIL7R for treatment of cancers.

Immuno-oncology is a rapidly growing field that holds great promise for patients with heretofore intractable cancers; however, the impact of immunotherapy has been limited by very low response rates in most cancers and individuals. Immunotherapies like immune checkpoint inhibitors have been shown to effectively eradicate different cancer types but unfortunately the response varies between patients and only a small fraction of patients reach remission. Accordingly, there is a need for the development of improved immunotherapies or drugs that can synergize with exiting immunotherapies to boost anti-cancer immunity. One such drug is those that increase expression of sIL7R, which, as a non-limiting example, could enhance the response level of a given patient and the response rates between patients.

Antisense oligonucleotide therapy targets a genetic sequence of a particular gene that is causative of a particular disease with a short oligonucleotide that is complementary to a target sequence. Typically, a single or double strand of nucleic acids is designed (DNA, RNA, a hybrid or a chemical analogue) that binds to a target sequence in a messenger RNA (mRNA), pre-mRNA, miRNA, non-coding RNA or other types RNAs to modulate the target RNA and induce a pharmacological response. In the case of splice-modulating antisense oligonucleotides (SM-ASOs), the complementary nucleic acid is designed to bind a specific sequence in a pre-mRNA that modifies the exon content of the resulting mRNA. Antisense oligonucleotides have been used to target diseases such as cancers, diabetes, amyotrophic lateral sclerosis (ALS), Duchenne muscular dystrophy, spinal muscular atrophy, Ataxia-telangiectasia, asthma, and arthritis, among others. Several antisense oligonucleotide drugs have been approved by the U.S. Food and Drug Administration (FDA), for the treatment for cytomegalovirus retinitis, homozygous familial hypercholesterolemia, Duchenne muscular dystrophy, and spinal muscular atrophy, to name a few, with the latter two being SM-ASOs. However, in each case the oligonucleotide target sequence must be tailored to the specific RNA underlying the disease in question or that its modulation could be therapeutic.

### SUMMARY OF THE INVENTION

In one embodiment, the present invention includes a method of treating a disease or condition with elevated levels of a soluble isoform of Interleukin 7 receptor (sIL7R) in a subject in need thereof, the method comprising: administering an effective amount of a composition comprising an oligonucleotide that specifically binds to a sequence of the Interleukin-7 receptor (IL7R) pre-mRNA that influences splicing of exon 6, wherein the oligonucleotide increases inclusion of exon 6 in IL7R pre-mRNAs and decreases expression of the soluble isoform of IL7R (sIL7R). In one aspect, the oligonucleotide is an antisense oligonucleotide (ASO) or a splice-modulating antisense oligonucleotide (SM-ASO). In another aspect, the oligonucleotide in the composition specifically binds to a sequence in IL7R pre-mRNA in at least one of the group consisting of an exonic splicing silencer (ESS) and/or an intronic splicing silencer (ISS), thereby enhancing inclusion of exon 6 in IL7R pre-mRNAs, and reducing expression of sIL7R. In another aspect, the oligonucleotide in the composition specifically binds to a sequence on IL7R pre-mRNA at intron-exon splice sites, branchpoint sequences, and/or polypyrimidine tracts. In another aspect, at least one or more nucleotide(s) in the oligonucleotide contains a non-naturally occurring modification comprising modifications or substitutions of: (1) the ribose or other sugar units, (2) bases, or (3) the backbone, selected from: one or more phosphorothioate, phosphorodithioate, phosphodiester, methyl phosphonate, phosphoramidate, methylphosphonate, phosphotriester, phosphoroaridate, morpholino, amidate carbamate, carboxymethyl, acetamidate, polyamide, sulfonate, sulfonamide, sulfamate, formacetal, thioformacetal, and/or alkylsilyl substitutions, partially or completely modified backbones, such as fully modified sugar phosphate backbone, a locked nucleic acid backbone, a peptidic backbone, a phosphotriester backbone, a phosphoramidate backbone, a siloxane backbone, a carboxymethylester backbone, an acetamidate backbone, a carbamate backbone, a thioether backbone, a bridged methylene phosphonate backbone, a phosphorothioate backbone, a methylphosphonate backbone, an alkylphosphonate backbone, a phosphate ester backbone, an alkylphosphonothioate backbone, a phosphorodithioate backbone, a carbonate backbone, a phosphate triester backbone, a carboxymethyl ester backbone, a methylphosphorothioate backbone, a phosphorodithioate backbone, a backbone having p-ethoxy linkages, sugar modifications such as 2′-O-methyl (2′-O-methylnucleotides), 2′-O-methyloxyethoxy (2′-O-MOE), a 2′-O-alkyl modified sugar moiety, or a bicyclic sugar moiety, nucleotide mimetics, peptide nucleic acids (PNA), morpholino nucleic acids, cyclohexenyl nucleic acids, anhydrohexitol nucleic acids, glycol nucleic acid, threose nucleic acid, and locked nucleic acids (LNA), and combinations of two or more of any of the foregoing.

In another aspect, at least one or more nucleotide(s) in an oligonucleotide is conjugated to a peptide, a cell penetrating peptide, an antibody, a nanobody, a camelid, an antibody variable region, a small molecule, and/or a ligand (including, a protein, a lipid, a carbohydrate) that enhances or can enhance the stability, distribution or delivery of an oligonucleotide to specific tissues.

In another aspect, at least one or more nucleotide(s) in the oligonucleotide contains a non-naturally occurring modification to the nucleotide bases. In another aspect, the oligonucleotide is selected from any of the SM-ASO SEQ IDs in Table 1 (SM-ASO SEQ ID NOS:1-13), or portions thereof, either alone or in combination, or a sequence having at least 70, 75, 80, 84, 85, 88, 92, 93, 94, 95, or 96% complementarity and or identity over the full target sequences within IL7R RNAs. In another aspect, the oligonucleotide targets any ofthe TARGET SEQ IDs in Table 2 (TARGET SEQ ID NOS:1-13), either fully or partially. In another aspect, the composition further comprises a pharmaceutically acceptable excipient, salts, or carrier. In another aspect, the disease or condition is an autoimmune disorder is selected from at least one of the following: multiple sclerosis, type I diabetes, rheumatoid arthritis, systemic lupus erythematosus, atopic dermatitis, ankylosing spondylitis, primary biliary cirrhosis, or inflammatory bowel syndromes such as ulcerative colitis, crohn's disease or any other conditions where sIL7R is elevated when compared to a normal subject without a disease or condition. In another aspect, the disease or condition is an inflammatory disease or condition. In another aspect, the oligonucleotide enhances the degradation of IL7R mRNAs that lack exon 6 by targeting an IL7R exon 5-exon 7 boundary, e.g., with ASOs, siRNAs, shRNAs that decrease stability of sIL7R RNA (e.g., increase degradation), and/or ASOs that decrease translation of sIL7R RNA. In another aspect, the method further comprises a combination therapy of the SM-ASO and one or more active agents effective for treating autoimmune diseases such as, but not limited to, mitoxatrone, interferon beta-1a, PEG-interferon beta-1a, azathioprine, fingolimod, natalizumab, methylprednisolone, or ocrelizumab. In another aspect, the method further comprises steps of obtaining cells from the patient and modifying the cells to transiently or permanently express the oligonucleotide that specifically binds to a sequence of the Interleukin-7 receptor (IL7R) pre-mRNA that influences splicing of exon 6. In another aspect, the method further comprises generating a vector that expresses the oligonucleotide that specifically binds to a sequence of the Interleukin-7 receptor (IL7R) pre-mRNA that influences splicing of exon 6 for use in gene therapy and treating the patient with the vector.

In another embodiment, the present invention includes a composition comprising an oligonucleotide that is an antisense oligonucleotide (ASO) or a splice-modulating antisense oligonucleotide (SM-ASO), that specifically binds to a sequence in pre-mRNAs of Interleukin 7 receptor (IL7R) that influences splicing of exon 6, wherein the SM-ASO increases inclusion of exon 6 in IL7R pre-mRNAs and decreases expression of the soluble isoform of IL7R (sIL7R). In one aspect, the oligonucleotide in the composition specifically binds to a sequence in IL7R pre-mRNA in at least one of the group consisting of an exonic splicing silencer (ESS) and/or an intronic splicing silencer (ISS), thereby enhancing inclusion of exon 6 in IL7R pre-mRNAs, and reducing expression of sIL7R. In another aspect, the oligonucleotide in the composition specifically binds to a sequence on IL7R pre-mRNA at intron-exon splice sites, branchpoint sequences, and/or polypyrimidine tracts. In another aspect, at least one or more nucleotide(s) in the oligonucleotide contains a non-naturally occurring modification comprising modifications or substitutions of: (1) the ribose or other sugar units, (2) bases, or (3) the backbone, selected from: one or more phosphorothioate, phosphorodithioate, phosphodiester, methyl phosphonate, phosphoramidate, methylphosphonate, phosphotriester, phosphoroaridate, morpholino, amidate carbamate, carboxymethyl, acetamidate, polyamide, sulfonate, sulfonamide, sulfamate, formacetal, thioformacetal, and/or alkylsilyl substitutions, partially or completely modified backbones, such as fully modified sugar phosphate backbone, a locked nucleic acid backbone, a peptidic backbone, a phosphotriester backbone, a phosphoramidate backbone, a siloxane backbone, a carboxymethylester backbone, an acetamidate backbone, a carbamate backbone, a thioether backbone, a bridged methylene phosphonate backbone, a phosphorothioate backbone, a methylphosphonate backbone, an alkylphosphonate backbone, a phosphate ester backbone, an alkylphosphonothioate backbone, a phosphorodithioate backbone, a carbonate backbone, a phosphate triester backbone, a carboxymethyl ester backbone, a methylphosphorothioate backbone, a phosphorodithioate backbone, a backbone having p-ethoxy linkages, sugar modifications such as 2′-O-methyl (2′-O-methylnucleotides), 2′-O-methyloxyethoxy (2′-O-MOE), a 2′-O-alkyl modified sugar moiety, or a bicyclic sugar moiety, nucleotide mimetics, peptide nucleic acids (PNA), morpholino nucleic acids, cyclohexenyl nucleic acids, anhydrohexitol nucleic acids, glycol nucleic acid, threose nucleic acid, and locked nucleic acids (LNA), and any combinations of two or more of any of the foregoing. In another aspect, at least one or more nucleotide(s) in the oligonucleotide contains a non-naturally occurring modification to the nucleotide bases. In another aspect, the oligonucleotide is selected from any of the SM-ASO SEQ IDs in Table 1 (SM-ASO SEQ ID NOS:1-13), or portions thereof, either alone or in combination, or a sequence having at least 70, 75, 80, 84, 85, 88, 92, 93, 94, 95, or 96% complementarity and or identity over the full target sequence within IL7R RNAs. In another aspect, the oligonucleotide targets any of the TARGET SEQ IDs in Table 2 (TARGET SEQ ID NOS:1-13), either fully or partially. In another aspect, the composition further comprises a pharmaceutically acceptable excipient, salts, or carrier. In another aspect, the composition is adapted for administration to treat an autoimmune disorder selected from at least one of the following: multiple sclerosis, type I diabetes, rheumatoid arthritis, systemic lupus erythematosus, atopic dermatitis, ankylosing spondylitis, primary biliary cirrhosis, inflammatory bowel syndromes such as ulcerative colitis and crohn's disease, or any other conditions where sIL7R is elevated. In another aspect, the oligonucleotide enhances the degradation of IL7R mRNAs that lack exon 6 by targeting an IL7R exon 5-exon 7 boundary, e.g., with ASOs, siRNAs, shRNAs that decrease stability of sIL7R RNA (e.g., increase degradation), and/or ASOs that decrease translation of sIL7R RNA.

In yet another embodiment, the present invention includes a method of increasing inclusion of exon 6 of an Interleukin-7 receptor (IL7R) pre-mRNA, the method comprising: contacting a splice modulating antisense oligonucleotide (SM-ASO) that specifically binds to a sequence of the Interleukin-7 receptor (IL7R) pre-mRNA that influences splicing of exon 6, wherein the SM-ASO increases inclusion of exon 6 in IL7R pre-mRNAs and decreases expression of the soluble isoform of IL7R (sIL7R). In one aspect, the SM-ASO in the composition specifically binds to a sequence in IL7R pre-mRNA in at least one of the group consisting of an exonic splicing silencer (ESS) and/or an intronic splicing silencer (ISS), thereby enhancing inclusion of exon 6 in IL7R pre-mRNAs, and reducing expression of sIL7R. In another aspect, the SM-ASO in the composition specifically binds to a sequence on IL7R pre-mRNA at intron-exon splice sites, branchpoint sequences, and/or polypyrimidine tracts. In another aspect, at least one or more nucleotide(s) in the SM-ASO contains a non-naturally occurring modification comprising modifications or substitutions of: (1) the ribose or other sugar units, (2) bases, or (3) the backbone, selected from: one or more phosphorothioate, phosphorodithioate, phosphodiester, methyl phosphonate, phosphoramidate, methylphosphonate, phosphotriester, phosphoroaridate, morpholino, amidate carbamate, carboxymethyl, acetamidate, polyamide, sulfonate, sulfonamide, sulfamate, formacetal, thioformacetal, and/or alkylsilyl substitutions, partially or completely modified backbones, such as fully modified sugar phosphate backbone, a locked nucleic acid backbone, a peptidic backbone, a phosphotriester backbone, a phosphoramidate backbone, a siloxane backbone, a carboxymethylester backbone, an acetamidate backbone, a carbamate backbone, a thioether backbone, a bridged methylene phosphonate backbone, a phosphorothioate backbone, a methylphosphonate backbone, an alkylphosphonate backbone, a phosphate ester backbone, an alkylphosphonothioate backbone, a phosphorodithioate backbone, a carbonate backbone, a phosphate triester backbone, a carboxymethyl ester backbone, a methylphosphorothioate backbone, a phosphorodithioate backbone, a backbone having p-ethoxy linkages, sugar modifications such as 2′-O-methyl (2′-O-methylnucleotides), 2′-O-methyloxyethoxy (2′-O-MOE), a 2′-O-alkyl modified sugar moiety, or a bicyclic sugar moiety, nucleotide mimetics, peptide nucleic acids (PNA), morpholino nucleic acids, cyclohexenyl nucleic acids, anhydrohexitol nucleic acids, glycol nucleic acid, threose nucleic acid, and locked nucleic acids (LNA), and any combinations of two or more of any of the foregoing. In another aspect, at least one or more nucleotide(s) in the SM-ASO contains a non-naturally occurring modification to the nucleotide bases. 5 In another aspect, the SM-ASO enhances the degradation of IL7R mRNAs that lack exon 6 by targeting an IL7R exon 5-exon 7 boundary, e.g., with ASOs, siRNAs, shRNAs that decrease stability of sIL7R RNA (e.g., increase degradation), and/or ASOs that decrease translation of sIL7R RNA. In another aspect, the SM-ASO blocks the translation of IL7R mRNAs that lack exon 6. In another aspect, the SM-ASO is selected from any of the SM-ASO SEQ IDs in Table 1 (SM-ASO SEQ ID NOS:1-13), or portions thereof either alone or in combination, or a sequence having at least 70, 75, 80, 84, 85, 88, 92, 93, 94, 95, or 96% complementarity and or identity over the full target sequence within IL7R RNAs. In another aspect, the oligonucleotide targets any of the TARGET SEQ IDs in Table 2 (TARGET SEQ ID NOS:1-13), either fully or partially. In another aspect, the composition further comprises a pharmaceutically acceptable excipient, salts, or carrier. In another aspect, the autoimmune disorder is selected from at least one of the following: multiple sclerosis, type I diabetes, rheumatoid arthritis, systemic lupus erythematosus, atopic dermatitis, ankylosing spondylitis, primary biliary cirrhosis, inflammatory bowel syndromes such as ulcerative colitis and crohn's disease, or any conditions where sIL7R is elevated. In another aspect, the method further comprises steps of obtaining cells from the patient and modifying the cells to transiently or permanently express the oligonucleotide that specifically binds to a sequence of the Interleukin-7 receptor (IL7R) pre-mRNA that influences splicing of exon 6.

In another aspect, the method further comprises generating a vector that expresses the oligonucleotide that specifically binds to a sequence of the Interleukin-7 receptor (IL7R) pre-mRNA that influences splicing of exon 6 for use in gene therapy and treating the patient with the vector.

In another embodiment, the present invention includes a composition for increasing inclusion of exon 6 in an Interleukin-7 receptor (IL7R) pre-mRNA, the method comprising: contacting a splice modulating antisense oligonucleotide (SM-ASO) that specifically binds to a sequence in the Interleukin-7 receptor (IL7R) pre-mRNA that influences splicing of exon 6, wherein the SM-ASO increases inclusion of exon 6 in IL7R pre-mRNAs and decreases expression of the soluble isoform of IL7R (sIL7R). In one aspect, the composition further comprises a combination therapy of the SM-ASO with one or more active agents effective for treating autoimmune diseases selected from, but not limited to, mitoxatrone, interferon beta-1a, PEG-interferon beta-1a, azathioprine, fingolimod, natalizumab, methylprednisolone, or ocrelizumab.

In another embodiment, the present invention includes a vector that expresses a nucleic acid comprising an oligonucleotide that is an antisense oligonucleotide (ASO), or a splice-modulating antisense oligonucleotide (SM-ASO), that specifically binds to a sequence in the Interleukin-7 receptor (IL7R) pre-mRNA that influences splicing of exon 6, wherein the SM-ASO increases inclusion of exon 6 in IL7R pre-mRNAs and decreases expression of the soluble isoform of IL7R (sIL7R). In one aspect, the vector is a viral vector or a plasmid.

In another embodiment, the present invention includes a vector that expresses a nucleic acid comprising an antisense oligonucleotide (ASO), splice-modulating antisense oligonucleotide (SM-ASO), translation-blocking antisense oligonucleotide, siRNA, shRNA or miRNA, that specifically binds a sequence in the Interleukin-7 receptor (IL7R) pre-mRNA that enhances inhibition or degradation of IL7R RNAs lacking exon 6, wherein the nucleic acid decreases expression of the soluble isoform of IL7R (sIL7R). In one aspect, the vector is a viral vector or a plasmid.

In another embodiment, the present invention includes a method of treating multiple sclerosis in a subject in need thereof, the method comprising: administering an effective amount of a composition comprising a splice modulating antisense oligonucleotide (SM-ASO) that specifically binds a sequence of an Interleukin-7 receptor (IL7R) pre-mRNA that influences splicing of exon 6, wherein the SM-ASO increases inclusion of exon 6 in IL7R pre-mRNAs and decreases expression of the soluble isoform of IL7R (sIL7R) in a pharmaceutically acceptable excipient. In one aspect, the method further comprises a combination therapy of the SM-ASO and one or more active agents effective for treating multiple sclerosis disease. In another aspect, the one or more agents for treating multiple sclerosis are selected from, but not limited to, mitoxatrone, interferon beta-1a, PEG-interferon beta-1a, azathioprine, fingolimod, natalizumab, methylprednisolone, or ocrelizumab.

In another embodiment, the present invention includes a method of treating a cancer in a subject in need thereof, the method comprising: administering an effective amount of a composition comprising an oligonucleotide that specifically binds a sequence of the Interleukin-7 receptor (IL7R) pre-mRNA that influences splicing of exon 6, wherein the oligonucleotide decreases inclusion of exon 6 in IL7R pre-mRNAs and increases expression of the soluble isoform of IL7R (sIL7R). In one aspect, the oligonucleotide is an antisense oligonucleotide (ASO) or a splice-modulating antisense oligonucleotide (SM-ASO). In another aspect, the oligonucleotide in the composition specifically binds to a sequence in IL7R pre-mRNA in at least one of the group consisting of an exonic splicing enhancer (ESE) and/or an intronic splicing enhancer (ISE), thereby decreasing inclusion of exon 6, and increasing expression of sIL7R. In another aspect, the oligonucleotide in the composition specifically binds to a sequence on IL7R pre-mRNA at intron-exon splice sites, branchpoint sequences, and/or polypyrimidine tracts, or any other element that influences splicing of exon 6, thereby decreasing inclusion of exon 6, and increasing expression of sIL7R. In another aspect, the oligonucleotide in the composition enhances the stability or translation of IL7R RNAs that lack exon 6 by binding to IL7R RNAs in the 5′UTR, 3′UTR, introns, exons, and/or their boundaries, thereby increasing expression of sIL7R. In another aspect, the oligonucleotide in the composition enhances the stability or translation of IL7R RNAs that lack exon 6 by binding to RNAs such as miRNAs, non-coding RNAs or other RNAs that regulate the stability of IL7R RNAs that lack exon 6, thereby increasing expression of sIL7R. In another aspect, at least one or more nucleotide(s) in the oligonucleotide contains a non-naturally occurring modification comprising modifications or substitutions of: (1) the ribose or other sugar units, (2) bases, or (3) the backbone, selected from: one or more phosphorothioate, phosphorodithioate, phosphodiester, methyl phosphonate, phosphoramidate, methylphosphonate, phosphotriester, phosphoroaridate, morpholino, amidate carbamate, carboxymethyl, acetamidate, polyamide, sulfonate, sulfonamide, sulfamate, formacetal, thioformacetal, and/or alkylsilyl substitutions, partially or completely modified backbones, such as fully modified sugar phosphate backbone, a locked nucleic acid backbone, a peptidic backbone, a phosphotriester backbone, a phosphoramidate backbone, a siloxane backbone, a carboxymethylester backbone, an acetamidate backbone, a carbamate backbone, a thioether backbone, a bridged methylene phosphonate backbone, a phosphorothioate backbone, a methylphosphonate backbone, an alkylphosphonate backbone, a phosphate ester backbone, an alkylphosphonothioate backbone, a phosphorodithioate backbone, a carbonate backbone, a phosphate triester backbone, a carboxymethyl ester backbone, a methylphosphorothioate backbone, a phosphorodithioate backbone, a backbone having p-ethoxy linkages, sugar modifications such as 2′-O-methyl (2′-O-methylnucleotides), 2′-O-methyloxyethoxy (2′-O-MOE), a 2′-O-alkyl modified sugar moiety, or a bicyclic sugar moiety, nucleotide mimetics, peptide nucleic acids (PNA), morpholino nucleic acids, cyclohexenyl nucleic acids, anhydrohexitol nucleic acids, glycol nucleic acid, threose nucleic acid, and locked nucleic acids (LNA), and combinations of two or more of any of the foregoing. In another aspect, at least one or more nucleotide(s) in the oligonucleotide contains a non-naturally occurring modification to the nucleotide bases. In another aspect, the oligonucleotide is selected from any of the SM-ASO SEQ IDs in Table 3 (SM-ASO SEQ ID NOS:14-50), or portions thereof, either alone or in combination, or a sequence having at least 70, 75, 80, 84, 85, 88, 92, 93, 94, 95, or 96% complementarity and or identity over the full target sequences within IL7R RNAs. In another aspect, the oligonucleotide targets any of the TARGET SEQ IDs in Table 4 (TARGET SEQ ID NOS:14-50), or portions thereof. In another aspect, the composition further comprises a pharmaceutically acceptable excipient, salts, or carrier. In another aspect, the method further comprises delivering recombinant sIL7R protein (in any form, including without limitation chimeras with Fc receptor) either alone or together with recombinant IL7 or other cytokines. In another aspect, the cancer demonstrates low response to conventional immunotherapy, as a non-limiting example, hepatocellular carcinoma. In another aspect, the method further comprises a combination therapy of an SM-ASO or recombinant sIL7R and one or more active agents effective for treating cancer such as, but not limited to, immune check point inhibitors (e.g., nivolumab, pembrolizumab, ipilimumab, atezolizumab, avelumab, durvalumab), therapeutic antibodies (e.g., trastuzumab, rituxumab, ramucirumab, bevacizumab), conventional chemotherapy (e.g., taxol), or therapeutic radiation. A list of active agents effective for treating cancer that can be used as part of a combination therapy of an SM-ASO or recombinant sIL7R is provided in FIG. 9. In another aspect, the method further comprises steps of obtaining cells from a patient and modifying the cells to transiently or permanently express the oligonucleotide that specifically binds to a sequence of the Interleukin-7 receptor (IL7R) pre-mRNA and decreases inclusion of exon 6, thereby increasing expression of sIL7R, or loading the cells with the ASO that enhances sIL7R expression and reintroducing the modified cells in the patient. In another aspect, the method further comprises steps of obtaining cells from a patient and modifying the cells to transiently or permanently express sIL7R and reintroducing the modified cells in the patient. In another aspect, the method further comprises generating a vector that expresses the oligonucleotide that specifically binds to a sequence of the Interleukin-7 receptor (IL7R) pre-mRNA that influences splicing of exon 6 for use in gene therapy and treating the patient with the vector. In another aspect, the method further comprises generating a vector that expresses sIL7R, thereby increasing expression of sIL7R, for use in gene therapy, and treating the patient with the vector.

In another embodiment, the present invention includes a composition comprising an oligonucleotide that is an antisense oligonucleotide (ASO) or a splice-modulating antisense oligonucleotide (SM-ASO), that specifically binds to a sequence in interleukin 7 receptor (IL7R) pre-mRNA that influences splicing of exon 6, wherein the SM-ASO decreases inclusion of exon 6 in IL7R pre-mRNAs and increases expression of the soluble isoform of IL7R (sIL7R). In another aspect, the oligonucleotide in the composition specifically binds to a sequence in IL7R pre-mRNA in at least one of the group consisting of an exonic splicing enhancer (ESE) and/or an intronic splicing enhancer (ISE), thereby decreasing inclusion of exon 6, and increasing expression of sIL7R. In another aspect, the oligonucleotide in the composition specifically binds to a sequence on IL7R pre-mRNA at intron-exon splice sites, branchpoint sequences, and/or polypyrimidine tracts, or any other element that influences splicing of exon 6, thereby decreasing inclusion of exon 6, and increasing expression of sIL7R. In another aspect, the oligonucleotide in the composition enhances the stability or translation of IL7R RNAs that lack exon 6 by binding to IL7R RNAs in the 5′UTR, 3′UTR, introns, exons, and/or their boundaries, thereby increasing expression of sIL7R. In another aspect, the oligonucleotide in the composition enhances the stability or translation of IL7R RNAs that lack exon 6 by binding to RNAs such as miRNAs, non-coding RNAs or other RNAs that regulate the stability of IL7R RNAs that lack exon 6, thereby increasing expression of sIL7R. In another aspect, at least one or more nucleotide(s) in the oligonucleotide contains a non-naturally occurring modification comprising modifications or substitutions of: (1) the ribose or other sugar units, (2) bases, or (3) the backbone, selected from: one or more phosphorothioate, phosphorodithioate, phosphodiester, methyl phosphonate, phosphoramidate, methylphosphonate, phosphotriester, phosphoroaridate, morpholino, amidate carbamate, carboxymethyl, acetamidate, polyamide, sulfonate, sulfonamide, sulfamate, formacetal, thioformacetal, and/or alkylsilyl substitutions, partially or completely modified backbones, such as fully modified sugar phosphate backbone, a locked nucleic acid backbone, a peptidic backbone, a phosphotriester backbone, a phosphoramidate backbone, a siloxane backbone, a carboxymethylester backbone, an acetamidate backbone, a carbamate backbone, a thioether backbone, a bridged methylene phosphonate backbone, a phosphorothioate backbone, a methylphosphonate backbone, an alkylphosphonate backbone, a phosphate ester backbone, an alkylphosphonothioate backbone, a phosphorodithioate backbone, a carbonate backbone, a phosphate triester backbone, a carboxymethyl ester backbone, a methylphosphorothioate backbone, a phosphorodithioate backbone, a backbone having p-ethoxy linkages, sugar modifications such as 2′-O-methyl (2′-O-methylnucleotides), 2′-O-methyloxyethoxy (2′-O-MOE), a 2′-O-alkyl modified sugar moiety, or a bicyclic sugar moiety, nucleotide mimetics, peptide nucleic acids (PNA), morpholino nucleic acids, cyclohexenyl nucleic acids, anhydrohexitol nucleic acids, glycol nucleic acid, threose nucleic acid, and locked nucleic acids (LNA), and any combinations of two or more of any of the foregoing. In another aspect, at least one or more nucleotide(s) in the oligonucleotide contains a non-naturally occurring modification to the nucleotide bases. In another aspect, the oligonucleotide is selected from any of the SM-ASO SEQ IDs in Table 3 (SM-ASO SEQ ID NOS:14-50), or portions thereof, either alone or in combination, or a sequence having at least 70, 75, 80, 84, 85, 88, 92, 93, 94, 95, or 96% complementarity and or identity over the full target sequence within IL7R RNAs. In another aspect, the oligonucleotide targets any of the TARGET SEQ IDs in Table 4 (TARGET SEQ ID NOS:14-50), either fully or partially. In another aspect, the composition further comprises a pharmaceutically acceptable excipient, salts, or carrier. In another aspect, the composition is adapted for administration to treat a cancer. In an embodiment, the composition comprising a therapeutic, may be formulated for either local or systemic delivery using topical, enteral or parenteral routes of administration. Additionally, an ASO or SM-ASO that is disclosed herein may be formulated by itself in a composition or in a composition where it is formulated together with one or more other therapeutics to create a single composition. In a further aspect, a composition comprising an ASO and/or SM-ASO is administered through an intravenous infusion, direct injection into the tumor microenvironment, a combination of both, orally, intrarectally, subcutaneously, intravaginally, intramuscularly, intrathecally or other commonly used routes of delivery.

In yet another embodiment, the present invention includes a method of decreasing inclusion of exon 6 in Interleukin-7 receptor (IL7R) pre-mRNA, the method comprising: contacting a splice modulating antisense oligonucleotide (SM-ASO) that specifically binds to a sequence of the Interleukin-7 receptor (IL7R) pre-mRNA that influences splicing of exon 6, wherein the SM-ASO decreases inclusion of exon 6 in IL7R pre-mRNAs and increases expression of the soluble isoform of IL7R (sIL7R). In another aspect, the oligonucleotide in the composition specifically binds to a sequence in IL7R pre-mRNA in at least one of the group consisting of an exonic splicing enhancer (ESE) and/or an intronic splicing enhancer (ISE), thereby decreasing inclusion of exon 6, and increasing expression of sIL7R. In another aspect, the SM-ASO in the composition specifically binds to a sequence on IL7R pre-mRNA at intron-exon splice sites, branchpoint sequences, and/or polypyrimidine tracts, or any other element that influences splicing of exon 6, thereby decreasing inclusion of exon 6, and increasing expression of sIL7R. In another aspect, the oligonucleotide in the composition enhances the stability or translation of IL7R RNAs that lack exon 6 by binding to IL7R RNAs in the 5′UTR, 3′UTR, introns, exons, and/or their boundaries, thereby increasing expression of sIL7R. In another aspect, the oligonucleotide in the composition enhances the stability or translation of IL7R RNAs that lack exon 6 by binding to RNAs such as miRNAs, non-coding RNAs or other RNAs that regulate the stability of IL7R RNAs that lack exon 6, thereby increasing expression of sIL7R. In another aspect, at least one or more nucleotide(s) in the SM-ASO contains a non-naturally occurring modification comprising modifications or substitutions of: (1) the ribose or other sugar units, (2) bases, or (3) the backbone, selected from: one or more phosphorothioate, phosphorodithioate, phosphodiester, methyl phosphonate, phosphoramidate, methylphosphonate, phosphotriester, phosphoroaridate, morpholino, amidate carbamate, carboxymethyl, acetamidate, polyamide, sulfonate, sulfonamide, sulfamate, formacetal, thioformacetal, and/or alkylsilyl substitutions, partially or completely modified backbones, such as fully modified sugar phosphate backbone, a locked nucleic acid backbone, a peptidic backbone, a phosphotriester backbone, a phosphoramidate backbone, a siloxane backbone, a carboxymethylester backbone, an acetamidate backbone, a carbamate backbone, a thioether backbone, a bridged methylene phosphonate backbone, a phosphorothioate backbone, a methylphosphonate backbone, an alkylphosphonate backbone, a phosphate ester backbone, an alkylphosphonothioate backbone, a phosphorodithioate backbone, a carbonate backbone, a phosphate triester backbone, a carboxymethyl ester backbone, a methylphosphorothioate backbone, a phosphorodithioate backbone, a backbone having p-ethoxy linkages, sugar modifications such as 2′-O-methyl (2′-O-methylnucleotides), 2′-O-methyloxyethoxy (2′-O-MOE), a 2′-O-alkyl modified sugar moiety, or a bicyclic sugar moiety, nucleotide mimetics, peptide nucleic acids (PNA), morpholino nucleic acids, cyclohexenyl nucleic acids, anhydrohexitol nucleic acids, glycol nucleic acid, threose nucleic acid, and locked nucleic acids (LNA), and any combinations of two or more of any of the foregoing. In another aspect, at least one or more nucleotide(s) in the SM-ASO contains a non-naturally occurring modification to the nucleotide bases.

In another aspect, the SM-ASO enhances the stability of IL7R mRNAs that lack exon 6 by targeting an IL7R exon 5-exon 7 boundary. In another aspect, the SM-ASO enhances the translation of IL7R mRNAs that lack exon 6. In another aspect, the SM-ASO is selected from any of the SM-ASO SEQ IDs in Table 3 (SM-ASO SEQ ID NOS:14-50), or portions thereof, either alone or in combination, or a sequence having at least 70, 75, 80, 84, 85, 88, 92, 93, 94, 95, or 96% complementarity and or identity over the full target sequence within IL7R RNAs. In another aspect, the oligonucleotide targets any of the TARGET SEQ IDs in Table 4 (TARGET SEQ ID NOS:14-50), either fully or partially. In another aspect, the composition further comprises a pharmaceutically acceptable excipient, salts, or carrier. In another aspect, the disorder is a type of cancer. In another aspect, the method further comprises steps of obtaining cells from a patient and modifying the cells to transiently or permanently express the oligonucleotide that specifically binds to a sequence of the Interleukin-7 receptor (IL7R) pre-mRNA and decreases inclusion of exon 6, thereby increasing expression of sIL7R, or loading the cells with the ASO that enhances sIL7R expression and reintroducing the modified cells in the patient. In another aspect, the method further comprises generating a vector that expresses the oligonucleotide that specifically binds to a sequence of the Interleukin-7 receptor (IL7R) pre-mRNA that influences splicing of exon 6 for use in gene therapy and treating the patient with the vector.

In another embodiment, the present invention includes a composition for increasing inclusion of exon 6 in an Interleukin-7 receptor (IL7R) pre-mRNA, the method comprising: contacting a splice modulating antisense oligonucleotide (SM-ASO) that specifically binds to a sequence in the Interleukin-7 receptor (IL7R) pre-mRNA that influences splicing of exon 6, wherein the SM-ASO decreases inclusion of exon 6 in IL7R pre-mRNAs and increases expression of the soluble isoform of IL7R (sIL7R). In one aspect, the composition further comprises a combination therapy of the SM-ASO with one or more active agents effective for treating cancer, such as, but not limited to, immune check point inhibitors (e.g., nivolumab), therapeutic antibodies (e.g., Herceptin), conventional chemotherapy (e.g., taxol), or therapeutic radiation.

In another embodiment, the present invention further comprises steps of obtaining cells from a patient and modifying the cells to transiently or permanently express the oligonucleotide that specifically binds to a sequence of the Interleukin-7 receptor (IL7R) pre-mRNA and decreases inclusion of exon 6, thereby increasing expression of sIL7R, or loading the cells with the ASO that enhances sIL7R expression and reintroducing the modified cells in the patient. In another aspect, the method further comprises steps of obtaining cells from the patient and modifying the cells to transiently or permanently express sIL7R and reintroduce the modified cells in the patient. In another embodiment, the present invention includes a vector that expresses the oligonucleotide that specifically binds to a sequence of the Interleukin-7 receptor (IL7R) pre-mRNA that influences splicing of exon 6 for use in gene therapy and treating the patient with the vector. In another embodiment, the present invention includes a vector that expresses sIL7R for use in gene therapy and treating the patient with the vector.

In another embodiment, the present invention includes a vector that expresses a nucleic acid comprising an oligonucleotide that is an antisense oligonucleotide (ASO), or a splice-modulating antisense oligonucleotide (SM-ASO), that specifically binds a sequence in the Interleukin-7 receptor (IL7R) pre-mRNA that influences splicing of exon 6, wherein the SM-ASO decreases inclusion of exon 6 in IL7R pre-mRNAs and increases expression of the soluble isoform of IL7R (sIL7R). In another embodiment, the present invention includes a vector that expresses sIL7R, thereby increasing expression of sIL7R. In one aspect, the vector is a viral vector or a plasmid.

### BRIEF DESCRIPTION OF THE DRAWINGS

For a more complete understanding of the features and advantages of the present invention, reference is now made to the detailed description of the invention along with the accompanying figures and in which:

FIGS. 1A to 1F. Screen of ASOs targeting functional cis-acting splicing elements within IL7R exon 6. (A) Schematics of GFP-IL7R reporter. A fluorescence reporter of IL7R exon 6 splicing was created by cloning the genomic sequence of IL7R spanning introns 5 and 6 and interrupting the coding sequence of GFP, wherein GFP is only expressed by exclusion of IL7R exon 6. (B) Schematics of approach comparing the effects of mutagenesis of functional cis-acting splicing elements to blocking of the corresponding elements with ASOs. Mutations to the 5′ss, ESE2 and ESS3 were introduced in the GFP-IL7R reporter and their effects were compared to steric blocking of the corresponding cis-element with ASOs. Expanded below is the sequence of exon 6 (light gray box), with relevant cis-acting elements indicated by bold horizontal lines: enhancer ESE2, silencers ESS2 and ESS3, and 5′ss. ESE2, ESS3 and the 5′ss were mutated by transversion substitutions and the mutated sequences are highlighted in a grey background box underneath each element: 5′Cons and 5′Mut represent consensus or crippling mutations to the 5′ss, respectively, whereas AESE2 and AESS3 represent mutations to ESE2 and ESS3, respectively. The sequence of ASOs targeting these cis-elements (IL7R-001—IL7R-005) is shown above the exon sequence: IL7R-001 blocks the 5′ss of exon 6, IL7R-002 blocks ESE2, IL7R-003 blocks ESS2 and ESS3, IL7R-004 blocks the sequence in between ESS2 and ESS3, and IL7R-005 blocks ESS3. (C, E) Reporter splicing. Splicing of exon 6 was quantified by RT-PCR in transcripts from the reporter in HeLa cells stably expressing wild-type or mutant versions of the reporter (C), or cells stably expressing the wild-type reporter and transfected with control (ASO-Ctrl) or experimental (IL7R-001—IL7R-005) morpholino ASOs at 10 μM with the Endo-Porter transfection reagent (E). Percentage (%) of exon 6 exclusion (mean±S.D.) was determined as: [excluded product/(excluded+included products)] and is shown under the gel images for each condition. (D, F) GFP expression. Expression of GFP was quantified as mean fluorescence intensity (MFI) by flow cytometry in the cells from panel C stably expressing the different versions of the reporter (D), or in cells from panel E transfected with ASOs (F). Quantification of GFP MFI (mean±S.D.) is shown as Log2(Fold-Change) [Log2(FC)] relative to wild-type for all mutants (D), or relative to control ASO for all ASOs (F). In all panels, statistical significance was assessed by Student's t-test pairwise comparison against wild-type or control ASO (* p<0.05, ** p<0.01, *** p<0.001).

FIGS. 2A to 2B. ASO-Walk screen to discover functional ASOs. Morpholino ASOs (15-25 nt) were complementary to overlapping sequences in IL7R introns 5 and 6 starting at the regions adjacent to exon 6 and moving away from the exon in 5 nt stretches. The ASOs were transfected at 10 μM into the HeLa cell line stably expressing the GFP-IL7R splicing reporter, in which GFP is expressed by exclusion of IL7R exon 6. A-B, GFP expression analysis of ASOs targeting introns 5 (A) or 6 (B). Changes in GFP expression were quantified as mean fluorescence intensity (MFI) by Flow Cytometry and are shown as Log2(fold-change) over control (ASO-Ctrl). The bars highlighted in gray indicate ASOs that enhanced GFP expression statistically significant. Statistical significance was assessed by Student's t test pairwise comparisons against control (* p<0.05, ** p<0.01, *** p<0.001).

FIGS. 3A to 3C. Concentration-dependent effect of lead ASOs on IL7R exon 6 splicing. Morpholino ASOs ASO-Ctrl, IL7R-001 or IL7R-004 were transfected with Endo-Porter into the wild-type reporter stable cell line at increasing concentrations (0, 1, 5 and 10 μM). Two days after transfection the cells were assayed for GFP expression and exon 6 splicing in transcripts from the reporter and the endogenous IL7R gene. (A) GFP expression. GFP mean fluorescence intensity (MFI) was quantified by flow cytometry. Quantification of MFI is shown as dose-response curves of Log2(FC) relative to 0 μM. (B-C) IL7R exon 6 splicing. Exon 6 exclusion was assessed in transcripts from the GFP-IL7R reporter (B) or the endogenous IL7R gene (C) by RT-PCR. Representative gel images are shown at the top with quantification of percentage (%) exon 6 exclusion (mean±S.D.) plotted at the bottom as a function of ASO concentration. Statistical significance was assessed in all panels by Student's t-test pairwise comparison against 0 μM (* p<0.05, ** p<0.01, *** p<0.001).

FIGS. 4A to 4B. Lead ASOs modulate sIL7R secretion in HeLa cells. Morpholino ASOs of interest (ASO-Ctrl, IL7R-001, IL7R-004, IL7R-005 and IL7R-006) were transfected at 10 μM into HeLa cells with the Endo-Porter transfection reagent, and the cells were assayed 3 days after for IL7R exon 6 splicing and sIL7R secretion. (A) Endogenous IL7R splicing. Percentage (%) of exon 6 exclusion in transcripts from the endogenous IL7R gene was quantified by RT-PCR and is shown as mean±S.D. for each ASO under the gel image. (B) Secretion of sIL7R. Levels of secreted sIL7R (mean±S.D.) were quantified by ELISA in supernatants from cells in panel A. Statistical significance in all panels was assessed by Student's t-test pairwise comparison against control (* p<0.05, ** p<0.01, *** p<0.001).

FIGS. 5A to 5C. Lead ASOs modulate expression of IL7R protein isoforms in human primary CD4⁺ T cells. Control (ASO-Ctrl) or lead (IL7R-001, IL7R-004, IL7R-005 and IL7R-006) morpholino ASOs were nucleofected at 2 μM into human primary CD4⁺ T cells. Cells were assayed for IL7R splicing, sIL7R secretion and mIL7R cell surface expression four days after nucleofection. (A) Endogenous IL7R splicing. Percentage (%) exon 6 exclusion was quantified in the endogenous IL7R transcripts by RT-PCR as before and the values (mean±S.D.) are shown for each ASO under the gel image. (B) Secretion of sIL7R. Levels of secreted sIL7R (mean±S.D.) were quantified by ELISA in supernatants from cells in panel A. (C) Cell surface expression of mIL7R. Staining of mIL7R was performed in intact cells and expression was quantified as mean fluorescence intensity (MFI) by flow cytometry. MFI values (mean±S.D.) are shown relative to ASO-Ctrl. Statistical significance was assessed in all panels by Student's t-test pairwise comparison against the control (* p<0.05, ** p<0.01, *** p<0.001).

FIGS. 6A to 6B. Pro-sIL7R ASOs phenocopy the enhanced exon 6 exclusion by the risk ‘C’ allele of the SNP rs6897932. Control (ASO-Ctrl) or lead pro-sIL7R morpholino ASOs IL7R-001 and IL7R-004 were transfected with Endo-Porter into HeLa cells stably expressing versions of the reporters containing either the risk ‘C’ allele or the protective ‘T’ allele of the IL7R SNP rs6897932 that promotes self-reactivity. (A) Reporter splicing. Percentage (%) of exon 6 exclusion in transcripts from the reporter was quantified by RT-PCR as before and the values (mean±S.D.) are plotted for each condition below the gel image. (B) GFP expression. GFP mean fluorescence intensity (MFI±S.D.) was quantified by flow cytometry as before and is shown for each condition relative to the C reporter treated with ASO-Ctrl. Statistical significance was assessed in all panels by Student's t-test pairwise comparison against the C reporter or as indicated (*** p<0.001).

FIGS. 7A to 7F. IL7R-001 enhances sIL7R secretion and IL7 signaling in CD4⁺ T cells. (A, B) Identification of Minimum Effective Concentration (MEC). Human primary CD4⁺ T cells were incubated for 3 days with increasing concentrations of control ASO (ASO-Ctrl) or IL7R-001 and assayed for IL7R exon 6 splicing by RT-PCR (A) and cell viability by flow cytometry with 7-AAD exclusion (B). The percentage (%) of exon 6 exclusion (% sIL7R RNA) was determined as before. The MEC (0.5 M, black arrow) was defined as the concentration that significantly enhanced sIL7R RNA to the target 50% exclusion without affecting cell viability. (C-D) Validation of MEC. Human primary CD4⁺ T cells were incubated for 3 days with 0 or 0.5 M (MEC) of either ASO-Ctrl or IL7R-001 ASOs and assayed for IL7R exon 6 splicing by RT-PCR (C) and sIL7R secretion in cell supernatants by ELISA (D). (E-F) Modulation of IL7 activity. Human primary CD4⁺ T cells were incubated with 0.5 M (MEC) of ASO-Ctrl or IL7R-001 ASOs for 2 days followed by treatment with 1 ng/ml IL7 for 1 day. IL7 was quantified in cell supernatants by ELISA (E) and the expression of IL7-induced genes BCL2 and CISH was quantified by RT-qPCR (F). Data in E & F are shown as Log2 of fold-change (Log2(FC)). Statistical significance was assessed by Student's t test pairwise comparisons to 0 M (A-B) or ASO-Ctrl (C-E).

FIGS. 8A to 8C. IL7R-001 potentiates IL7 and enhances T cell survival. Human primary CD4⁺ T cells were incubated with 0.5 M of ASO-Ctrl or IL7R-001, followed by treatment with 10 ng/ml IL7. On days 0 and 5 post IL7 treatment, the cells were assayed for (A) IL7 activity by RT-qPCR quantification of the IL7-induced gene BCL2, (B) sIL7R secretion by ELISA in cell supernatants, and (C) cell survival by flow cytometry with 7-AAD exclusion.

FIG. 9. A listing of therapeutics used to treat a cancer that can be used with an ASO or SM-ASO of the present invention to treat a patient suffering from a cancer.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not limit the invention, except as outlined in the claims.

The present invention is directed to novel compositions and methods that reduce or increase soluble IL7R (sIL7R) to treat autoimmune diseases (e.g., multiple sclerosis) or cancer, respectively. The present invention uses SM-ASOs to control alternative splicing of the Interleukin 7 receptor (IL7R) pre-mRNAs, either to prevent or diminish expression of sIL7R or the opposite to increase expression of sIL7R. For example, given the ability of sIL7R to enhance self-reactivity it is demonstrated herein that increasing sIL7R levels enhances response to currently employed immunotherapies (e.g., immune check point inhibitors).

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, organic synthesis, nucleic acid chemistry and nucleic acid hybridization are those well-known and commonly employed in the art. Further, standard techniques can be used for nucleic acid and peptide synthesis. Such techniques and procedures are generally performed according to conventional methods known in the art and from various general references (e.g., Sambrook and Russell, 2012, Molecular Cloning, A Laboratory Approach, Cold Spring Harbor Press, Cold Spring Harbor, N.Y., and Ausubel et al., 2012, Current Protocols in Molecular Biology, John Wiley & Sons, NY), relevant portions incorporated herein by reference.

Conventional notations are used herein to describe polynucleotide sequences, e.g., the left-hand end of a single-stranded polynucleotide sequence is the 5′-end and vice versa for the 3′-end (right-hand end); the left-hand direction of a double-stranded polynucleotide sequence is referred to as the 5′-direction and vice versa for the 3′-direction (right-hand direction), with regard to sequences, such as those that become coding sequences. The direction of 5′ to 3′ addition of nucleotides to nascent RNA transcripts is referred to as the transcription direction. The DNA strand having the same sequence as an mRNA is referred to as the “coding strand”. Sequences on the DNA or RNA strand that are located 5′ to a reference point on the DNA or RNA are referred to as “upstream sequences”, and sequences on the DNA or RNA strand that are 3′ to a reference point on the DNA or RNA are referred to as “downstream sequences.”

As used herein, the term “antisense” refers to an oligonucleotide having a sequence that hybridizes to a target sequence in RNA by Watson-Crick base pairing, to form an RNA:oligonucleotide heteroduplex with the target sequence, typically with an mRNA or pre-mRNA. The antisense oligonucleotide may have exact sequence complementarity and or identity to the target sequence or near complementarity and or identity. These antisense oligonucleotides may block or inhibit translation of the mRNA, modify the processing of an mRNA to produce a splice variant of the mRNA, and/or promote specific degradation of a given mRNA or variant of an mRNA. One non-limiting example can also be RNase H dependent degradation. It is not necessary that the antisense sequence be complementary solely to the coding portion of the RNA molecule. The antisense sequence may be complementary to regulatory sequences specified on the non-coding region of an RNA molecule (e.g. introns, untranslated regions) encoding a protein, which regulatory sequences control expression of the coding sequences. Antisense oligonucleotides are typically between about 5 to about 100 nucleotides in length, more typically, between about 7 and about 50 nucleotides in length, and even more typically between about 10 nucleotides and about 30 nucleotides in length.

As used herein, the term “nucleic acid” or a “nucleic acid molecule” refer to any DNA or RNA molecule, either single or double stranded, whether in linear or circular form. With reference to nucleic acids of the present invention, the term “isolated nucleic acid”, when applied to DNA or RNA, refers to a DNA or RNA molecule that is separated from sequences with which it is immediately contiguous in the naturally occurring genome or gene products of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

As used herein, the terms “specifically hybridizing” or “substantially complementary” refer to the association between two nucleotide molecules of sufficient complementarity and or identity to permit hybridization under pre-determined conditions generally used in the art. Examples of low, middle or intermediate and high stringency hybridization conditions are well known to the skilled artisan, e.g., using Sambrook and Russell, 2012, Molecular Cloning, A Laboratory Approach, Cold Spring Harbor Press, Cold Spring Harbor, N.Y., or Ausubel et al., 2012, Current Protocols in Molecular Biology, John Wiley & Sons, NY, relevant portions incorporated herein by reference.

As used herein, the phrase “chemically modified oligonucleotide” refers to a short nucleic acid (DNA or RNA) that can be a sense or antisense that includes modifications or substitutions, such as those taught by Wan and Seth, “The Medicinal Chemistry of Therapeutic Oligonucleotides”, J. Med. Chem. 2016, 59, 21, 9645-9667, relevant portions incorporated wherein, which may include modifications of: (1) the ribose or other sugar units, (2) bases, or (3) the backbone, which in nature is composed of phosphates, as are known in the art. Non-limiting examples of modifications or nucleotide analogs include, without limitation, nucleotides with phosphate modifications comprising one or more phosphorothioate, phosphorodithioate, phosphodiester, methyl phosphonate, phosphoramidate, methylphosphonate a peptide, a cell penetrating peptide, an antibody, a nanobody, a camelid, an antibody variable region, a small molecule, and/or a ligand (including, a protein, a lipid, a carbohydrate) that enhances or can enhance the stability, distribution or delivery of an oligonucleotide to specific tissues, phosphotriester, phosphoroaridate, morpholino, amidate carbamate, carboxymethyl, acetamidate, polyamide, sulfonate, sulfonamide, sulfamate, formacetal, thioformacetal, and/or alkylsilyl substitutions (see, e.g., Hunziker and Leumann (1995) Nucleic Acid Analogues: Synthesis and Properties, in Modern Synthetic Methods, VCH, 331-417; Mesmaeker et al. (1994) Novel Backbone Replacements for Oligonucleotides, in Carbohydrate Modifications in Antisense Research, ACS, 24-39); nucleotides with modified sugars (see, e.g., U.S. Patent Application Publication No. 2005/0118605) and sugar modifications such as 2′-O-methyl (2′-O-methylnucleotides) and 2′-O-methyloxyethoxy (2′-O-MOE), a 2′-O-alkyl modified sugar moiety, or a bicyclic sugar moiety, and nucleotide mimetics such as, without limitation, peptide nucleic acids (PNA), morpholino nucleic acids, cyclohexenyl nucleic acids, anhydrohexitol nucleic acids, glycol nucleic acid, threose nucleic acid, and locked nucleic acids (LNA), as well as partially or completely modified backbones, such as fully modified sugar phosphate backbone, a locked nucleic acid backbone, a peptidic backbone, a phosphotriester backbone, a phosphoramidate backbone, a siloxane backbone, a carboxymethylester backbone, an acetamidate backbone, a carbamate backbone, a thioether backbone, a bridged methylene phosphonate backbone, a phosphorothioate backbone, a methylphosphonate backbone, an alkylphosphonate backbone, a phosphate ester backbone, an alkylphosphonothioate backbone, a phosphorodithioate backbone, a carbonate backbone, a phosphate triester backbone, a carboxymethyl ester backbone, a methylphosphorothioate backbone, a phosphorodithioate backbone, a backbone having p-ethoxy linkages, and a combinations of two or more of any of the foregoing (see, e.g., U.S. Pat. Nos. 5,886,165; 6,140,482; 5,693,773; 5,856,462; 5,973,136; 5,929,226; 6,194,598; 6,172,209; 6,175,004; 6,166,197; 6,166,188; 6,160,152; 6,160,109; 6,153,737; 6,147,200; 6,146,829; 6,127,533; and 6,124,445 and Wan and Seth, “The Medicinal Chemistry of Therapeutic Oligonucleotides”, J. Med. Chem. 2016, 59, 21, 9645-9667, relevant portions incorporated herein by reference).

As used herein, the term “expression cassette” refers to a nucleic acid molecule comprising a coding sequence operably linked to promoter/regulatory sequences necessary for transcription, processing and, optionally, translation or splicing of the coding sequence.

The IL7R SM-ASOs that decrease sIL7R can be used for the treatment of diseases or disorders such as autoimmune and/or inflammatory diseases. The IL7R SM-ASOs that increase sIL7R can be used for immuno-oncology applications. Whether increasing or decreasing the expression of sIL7R message or protein, the present invention can be used in conjunction with gene therapy and ex vivo applications. For example, the oligonucleotides can be used in a method in which cells are isolated from the subject or another subject, and the cells are modified to express transiently or permanently the oligonucleotides that modify the expression of sIL7R, or the cells could be loaded with the oligonucleotides that modify expression of sIL7R, and the cells can then be transferred back into the subject. The present invention can be used with the various known methods of delivery and expression, such as plasmid or viral vectors. Also, the present invention can be used with all methods for modification of cells, e.g., gene editing, delivery of nucleic acids (any nucleic acid, either natural, synthetic or modified), proteins (full-length protein or peptides), whether transient or permanent, or under the control of regulatable promoters. The oligonucleotide or vectors that express the oligonucleotides can be delivered via known methods, such as the following non-limiting examples, transfection, electroporation, carrier-mediated, viral, free uptake, etc.

As used herein, the term “promoter/regulatory sequence” refers to a nucleic acid sequence that is required for expression of a gene product operably linked to the promoter/regulator sequence. In some instances, the promoter/regulatory sequence may be the core promoter sequence and in other instances, this sequence may also include an enhancer sequence and other regulatory elements that are required for expression of the gene product. The promoter/regulatory sequence may be, for example, a sequence that drives the expression of a gene product in a constitutive and/or inducible manner. As used herein, the term “inducible promoter” refers to a nucleotide sequence which, when operably linked with a polynucleotide which encodes or specifies a gene product, causes the gene product to be produced substantially only when an inducer which corresponds to the promoter is present.

As used herein, the terms “percent similarity”, “percent identity” and “percent homology”, when referring to a comparison between two specific sequences, identify the percentage or bases that are the same along a particular sequence. The percentage of similarity, identity or homology can be calculated using, e.g., the University of Wisconsin GCG software program or equivalents.

As used herein, the term “percent complementarity or identity”, when referring to a comparison between two complementary sequences, such as an antisense oligonucleotide and its target sequence, identify the percentage or bases that are complementary between the antisense oligonucleotide and the target sequence.

As used herein, the term “replicon” refers to any genetic element, for example, a plasmid, cosmid, bacmid, phage or virus, which is capable of replication largely under its own control. A replicon may be either RNA or DNA and may be single or double stranded.

As used herein, the term “vector” refers to a genetic element, such as a plasmid, cosmid, bacmid, phage or virus, to which another genetic sequence or element (either DNA or RNA) may be attached. The vector may be a replicon so as to bring about the replication of the attached sequence or element. An “expression vector” is a vector that facilitates the expression of a nucleic acid, such as an oligonucleotide, or a polypeptide coding nucleic acid sequence in a host cell or organism.

As used herein, the term “operably linked” refers to a nucleic acid sequence placed into a functional relationship with another nucleic acid sequence. Examples of nucleic acid sequences that may be operably linked include, without limitation, promoters, transcription terminators, enhancers or activators and heterologous genes which when transcribed and, if appropriate to, translated will produce a functional product such as a protein, ribozyme or RNA molecule.

As used herein, the term “oligonucleotide,” refers to a nucleic acid strand, single or double stranded that has a length that is, typically, less than a coding sequence for a gene, e.g., the oligonucleotide will generally be at least 4-6 bases or base-pairs in length, and up to about 200, with the most typical oligonucleotide being in the range of 8-20, 10-25, 12-30, or about 30, 35, 40, or 50 bases or base-pairs.

In one specific example of the present invention, the oligonucleotide is a nucleic acid strand having a sequence that modulates the inclusion of exon 6 in pre-mRNAs of the Interleukin-7 receptor (IL7R) gene and is defined as a nucleic acid molecule comprised of two or more ribo or deoxyribonucleotides, preferably more than four, and can include diverse chemical modifications and/or non-naturally occurring nucleotides. The exact size and chemical composition of the oligonucleotide will depend on various factors and on the particular application and use of the oligonucleotide, which can be varied as will be known to the skilled artisan without undue experimentation following the teachings herein and as taught in, e.g., Sambrook and Russell, 2012, Molecular Cloning, A Laboratory Approach, Cold Spring Harbor Press, Cold Spring Harbor, N.Y., or Ausubel et al., 2012, Current Protocols in Molecular Biology, John Wiley & Sons, NY, relevant portions incorporated herein by reference.

As used herein, the term “splice variant or isoform of an mRNA”, is meant a variant mRNA, which could be defective or pathogenic and be the result of alternative splicing of the RNA encoding a protein. Splicing events that produce a splice variant of the mRNA that is defective or leads to pathology will be referred in the present invention as a splicing defect. One example of such a splicing defect is the exclusion of exon 6 of IL7R causing expression of a shorter protein, soluble IL7R (sIL7R), which is secreted from the cell, leading to its presence in, e.g., the bloodstream or other bodily fluids. The present invention targets the elements (i.e., sequences) within IL7R pre-mRNAs that control the inclusion or exclusion of IL7R exon 6 in the final mature or processed mRNA, or sequences within sIL7R mRNA that control its translation or stability.

As used herein, the term “splice variant or isoform of a protein”, is meant a variant protein, which could be defective or pathogenic and be the result of alternative splicing of an RNA encoding a protein. Alternatively, when discussing those variants that increase degradation, those splice variants would reduce or eliminate protein production. Splicing events that produce a splice variant of a protein that is defective or leads to pathology will be referred in the present invention as a splicing defect. One example of such a splicing defect is the exclusion of exon 6 of IL7R causing expression of a shorter protein, soluble IL7R (sIL7R), which is secreted from the cell, leading to its presence in, e.g., the bloodstream or other bodily fluids. The present invention targets the elements (i.e., sequences) within IL7R pre-mRNAs that control the inclusion or exclusion of IL7R exon 6 in the final mature or processed mRNA, or sequences within sIL7R mRNA that control its translation or stability.

As used herein, the term “treatment”, refers to reversing, alleviating, delaying the onset of, inhibiting the progress of, and/or preventing a disease or disorder, including a cancer or one or more symptoms thereof, to which the term is applied in a subject, e.g., an autoimmune disease or disorder, or a cancer. In some embodiments, the treatment may be applied after one or more symptoms have developed. In other embodiments, treatment may be administered in the absence of symptoms. For example, treatment may be administered prior to symptoms (e.g., in light of a history of symptoms, family disease history and/or one or more other susceptibility factors), or after symptoms have resolved, for example to prevent or delay their reoccurrence. One such non-limiting example is relapsing-remitting MS.

As used herein, the terms “effective amount” and “pharmaceutically effective amount” refer to a sufficient amount of an agent to provide the desired biological result. Preferably, the sufficient amount of the agent does not induce toxic side effects. The present invention using IL7R SM-ASOs that reduce sIL7R should lead to a reduction and/or alleviation of the signs, symptoms, or causes of autoimmune diseases or disorders. As designed, the present invention is not expected to cause a reduction in the host immune response, and thus have few or low side effects associated with broad immunosuppression. An appropriate effective amount in any individual case may be determined by one of ordinary skill in the art using routine experimentation. The present invention using IL7R SM-ASOs that increase sIL7R should lead to a reduction and/or alleviation of the signs, symptoms, or causes of cancers. As designed, the present invention is expected to cause an enhancement in the host immune response as a monotherapy and/or to enhance current immunotherapies as a combination therapy. An appropriate effective amount in any individual case may be determined by one of ordinary skill in the art using routine experimentation.

The present invention may be provided in conjunction with one or more “pharmaceutically acceptable” agents, carriers, buffers, salts, or other agents listed in the U.S. Pharmacopeia or other generally recognized pharmacopeia for use in animals, and more particularly in humans, which generally indicates approval by a regulatory agency of the Federal government or a state government. Typical pharmaceutically acceptable formulations for use with oligonucleotides include but are not limited to salts such as: calcium chloride dihydrate (US Pharmacopeia (USP)), magnesium chloride hexahydrate USP, potassium chloride USP, sodium chloride USP; and may include buffers such as” sodium phosphate dibasic anhydrous USP, sodium phosphate monobasic dihydrate USP, and water USP.

Typically, the pH of the product may be modified using hydrochloric acid or sodium hydroxide to a pH of ˜6.8, 6.9, 7.0, 7.1, or 7.2.

The present invention may be provided in conjunction with one or more diagnostic tests that are used to demonstrate the effectiveness of the treatment.

As used herein, the term “carrier” refers to, for example, a diluent, preservative, solubilizer, emulsifier, adjuvant, excipient, auxiliary agent or vehicle with which an active agent of the present invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions may be employed as carriers. Suitable pharmaceutical carriers are described in “Remington's Pharmaceutical Sciences” 2012.

In one embodiment, the present invention is directed to novel compositions and methods to treat autoimmune disorders, including but not limited to Multiple Sclerosis (MS). MS is the most common neurological disease of early adulthood and is mediated by autoimmune mechanisms that lead to demyelination and neuronal damage in the central nervous system, resulting in progressive neurological dysfunction. To date, there is no cure for the disease and current available treatments focus on preventing future immunological attacks, often by suppressing the immune system. This immunosuppressive approach causes a plethora of adverse side effects, among them increased risk of cancer and infections that could be severe or lethal. Accordingly, the present inventors have developed novel therapeutics to meet the clear, unmet need for the development of effective and well-tolerated therapies to arrest MS development, without adverse immunosuppressive side effects.

The present invention is also directed to novel compositions and methods to treat cancer. Cancer is the second cause of death in the United States and is mediated by many etiologies. To date, many cancers cannot be treated; however, recently therapies based on immune recognition and killing of cancer cells have led to new hope. Unfortunately, many patients receiving immunotherapy do not respond well and some cancer types (e.g., hepatocellular carcinoma) have low response rates. Accordingly, the present inventors have developed novel therapeutics to meet the clear, unmet need for improved immunotherapies or drugs that can enhance the effectiveness of conventional immunotherapies.

The present inventors have developed targeted antisense oligonucleotides, such as antisense oligonucleotides (ASOs) and/or splice-modulating antisense oligonucleotides (SM-ASOs), to correct a specific MS etiology. SM-ASOs have proven to be a novel and valuable therapeutic tool to treat disorders caused by aberrant RNA splicing. Three such SM-ASOs have received recent approvals by the FDA for treatment of Spinal Muscular Atrophy (Spinraza, Biogen) and Duchenne Muscular Dystrophy (Exondys 51 and Vyondys 53, Sarepta Therapeutics).

The novel targeted therapeutics correct aberrant splicing of the interleukin 7 receptor (IL7R) RNAs, where exclusion of the alternative exon 6 leads to elevated levels of the pathogenic soluble form of the IL7R (sIL7R). Several lines of evidence directly link and strongly support a role for alternative splicing of IL7R exon 6 in the pathogenesis of MS and other autoimmune diseases: (1) genetic variants that increase exclusion of this exon are strongly associated with increased MS risk (Galarza-Munoz et al., 2017; Gregory et al., 2007); (2) sIL7R exacerbates the severity and progression of the disease in the Experimental Autoimmune Encephalomyelitis (EAE) mouse model of MS (Lundstrom et al., 2013); (3) sIL7R potentiates the bioavailability and activity of IL7 leading to enhance homeostatic expansion of T cells (Lundstrom et al., 2013), and (4) sIL7R is elevated in patients from several autoimmune diseases including MS, type I diabetes, rheumatoid arthritis and systemic lupus erythematosus (Badot et al., 2011; Badot et al., 2013; Lauwerys et al., 2014; McKay et al., 2008; Monti et al., 2013).

The present inventors have developed several SM-ASOs (Table 1), among them the lead ASOs IL7R-005 and IL7R-006, that promote inclusion of exon 6 in IL7R pre-mRNAs and correct expression of IL7R protein isoforms in cultured cells. Critical to this therapeutic approach, these SM-ASOs decrease sIL7R levels without a negative impact on expression of the membrane-bound IL7R (mIL7R). To treat autoimmunity, the present invention is used to block specific sequences in IL7R pre-mRNAs that drive exon 6 exclusion, thereby promoting exon 6 inclusion and reducing sIL7R levels. Additionally, to treat cancer, the present invention is used to block specific sequences in IL7R pre-mRNAs that drive exon 6 inclusion, thereby decreasing exon 6 inclusion and increasing sIL7R levels. The skilled artisan will recognize that the SM-ASOs of the present invention can be used alone or in combination with other therapies. Furthermore, through simple single base mutation, the SM-ASOs can be adapted to control splicing of exon 6 in allelic variants of human IL7R, or of IL7R in different animals, or in individuals carrying polymorphisms or where mutations have occurred at the targeted sequences, thus tailoring the complementarity and or identity of the SM-ASOs to the variant sequences.

TABLE 1
Sequence of SM-ASOs that reduce sIL7R (anti-sIL7R SM-ASOs).

| | | | Disease | SEQ |
|---|---|---|---|---|
| SM-ASO | SM-ASO Sequence* | Efficiency\hat{ } | application | ID NO: |
| IL7R-005 | GACCAACAGAGCGAC | +++ | Autoimmunity | 1 |
| IL7R-006 | CACAAUCACCCUCUUUAUUA | +++ | Autoimmunity | 2 |
| IL7R-008 | CGUGAUCCCACACAA | +++ | Autoimmunity | 3 |
| (Cluster 2) | | | | |
| IL7R-009 | CUGUCCGUGAUCCCA | +++ | Autoimmunity | 4 |
| (Cluster 2) | | | | |
| IL7R-010 | UCUGACUGUCCGUGA | +++ | Autoimmunity | 5 |
| (Cluster 2) | | | | |
| IL7R-066 | UCUGACUGUCCGUGAU | +++ | Autoimmunity | 6 |
| (Entire | CCCACACAA | | | |
| sequence of | | | | |
| Cluster 2) | | | | |
| IL7R-011 | GCUUAAGCUCUGACUGUC | + | Autoimmunity | 7 |
| IL7R-017 | UUUGUGGUUUUCUCA | ++ | Autoimmunity | 8 |
| IL7R-024 | CAUGCAGUGGAGGUA | + | Autoimmunity | 9 |
| IL7R-025 | AGUGGAGGUAGGGUC | +++ | Autoimmunity | 10 |
| (Cluster 1) | | | | |
| IL7R-026 | GGGUAGGGUCUCAGG | +++ | Autoimmunity | 11 |
| (Cluster 1) | | | | |
| IL7R-027 | GGGUCUCAGGGUGCU | +++ | Autoimmunity | 12 |
| (Cluster 1) | | | | |
| IL7R-041 | AGUGGUGGUAGGGUCU | +++ | Autoimmunity | 13 |
| (Entire | CAGGGUGCU | | | |
| sequence of | | | | |
| Cluster 1) | | | | |

*Nucleotides highlighted in bold and underlined indicate positions where a mismatch was engineered in the SM-ASO sequence to disrupt potential secondary structures that could limit the activity of the SM-ASO. Uridines (U) are replaced with thymidines (T) in DNA ASOs or DNA-RNA hybrid ASOs.
\hat{ }Efficacy scale: low (+), intermediate (++), and high (+++).

TABLE 2
Target sequence of SM-ASOs that reduce sIL7R
(anti-sIL7R SM-ASOs).

| | | | | SEQ |
|---|---|---|---|---|
| | | | Disease | ID |
| SM-ASO | Target Sequence* | Efficiency\hat{ } | application | NO: |
| IL7R-005 | GUCGCUCUGUUGGUC | +++ | Autoimmunity | 1 |
| IL7R-006 | UAAUAAAGAGGGUGA | +++ | Autoimmunity | 2 |
| | UUGUG | | | |
| IL7R-008 | UUGUGUGGGAUCACG | +++ | Autoimmunity | 3 |
| (Cluster 2) | | | | |
| IL7R-009 | UGGGAUCACGGACAG | +++ | Autoimmunity | 4 |
| (Cluster 2) | | | | |
| IL7R-010 | UCACGGACAGUCAGA | +++ | Autoimmunity | 5 |
| (Cluster 2) | | | | |
| IL7R-066 | UUGUGUGGGAUCACG | +++ | Autoimmunity | 6 |
| (Entire | GACAGUCAGA | | | |
| sequence of | | | | |
| Cluster 2) | | | | |
| IL7R-011 | GACAGUCAGAGCUUAA | + | Autoimmunity | 7 |
| | GC | | | |
| IL7R-017 | UGAGAAAACCACAAA | ++ | Autoimmunity | 8 |
| IL7R-024 | UACCCCCACUGCAUG | + | Autoimmunity | 9 |
| IL7R-025 | GACCCUACCCCCACU | +++ | Autoimmunity | 10 |
| (Cluster 1) | | | | |
| IL7R-026 | CCUGAGACCCUACCC | +++ | Autoimmunity | 11 |
| (Cluster 1) | | | | |
| IL7R-027 | AGCACCCUGAGACCC | +++ | Autoimmunity | 12 |
| (Cluster 1) | | | | |
| IL7R-041 | AGCACCCUGAGACCC | +++ | Autoimmunity | 13 |
| (Entire | UACCCCCACU | | | |
| sequence of | | | | |
| Cluster 1) | | | | |

* Nucleotides highlighted in bold and underlined indicate positions where a mismatch was engineered at the complementary position in the SM-ASO to disrupt potential secondary structures that could limit the activity of the SM-ASO.
\hat{ }Efficacy scale: low (+), intermediate (++), and high (+++).

A wide variety of SM-ASOs can be used with the present invention, e.g., those that include a wide variety of base, sugar or backbone modifications to the SM-ASOs. Non-limiting examples of SM-ASOs can include native nucleic acids, but can also include, e.g., backbone or base modifications (chemically modified oligonucleotides) that increase the efficiency of binding of the SM-ASO, increase the stability (e.g., half-life) of the SM-ASO, increase its expression, control where its expressed, distributed or localized, and the like.

Current treatments for autoimmune diseases, such as MS, have helped autoimmune disease patients manage their symptoms, yet these drugs are far from ideal given the wide variety of adverse side effects they cause, which can be severe or lethal. The development of effective but yet safer MS drugs has been hindered by the complex nature of the disease, wherein a multitude of etiologies lead to MS pathogenesis. Given that all these etiologies culminate in a breach of immunological tolerance against myelin, the field has, so far, focused on developing therapies to diminish immunological responses via diverse mechanisms. For example, natalizumab is designed to block migration of leukocytes across blood-brain barrier and their recruitment to sites of inflammation. Another example is ocrelizumab, which depletes B-cells. However, both of these mechanisms (although through different actions) ultimately lead to immunosuppression. In order to provide effective yet safer drugs to the patients, instead of dealing with the consequences of a given etiology via immune modulation, it is imperative to develop new therapies targeting correction of specific MS etiologies, which the present inventors refer to herein as immune correction.

The canonical, membrane-bound interleukin 7 receptor (mIL7R) has been a previous candidate of therapeutic intervention in MS and numerous autoimmune disorders. However, mIL7R is crucial for T cell homeostasis and normal immune function, and loss of IL7R function in both human and mouse cause severe immunodeficiency (Maraskovsky et al., 1996; Peschon et al., 1994; Puel et al., 1998; Roifman et al., 2000). Accordingly, novel MS therapies that inhibit expression or function of mIL7R would cause severe immunodeficiency. The therapeutic SM-ASOs developed here correct aberrant splicing of IL7R exon 6, and in doing so, they diminish sIL7R levels while preventing a negative impact on mIL7R expression and/or function. Accordingly, unlike current MS treatments relying on immunosuppressive mechanisms, the therapeutic SM-ASOs of the present invention that reduce sIL7R (Tables 1 & 2) are an effective and safer option to treat MS that avoid immunosuppression. The SM-ASOs of the present invention that reduce sIL7R (Tables 1 & 2) represent a major improvement over current drugs in that they correct the root of the problem rather than deal with the consequences of it by suppressing the immune system.

Furthermore, increased levels of sIL7R (when compared to the normal levels of sIL7R in subject that does not have an autoimmune disease) has been associated with other autoimmune diseases such as type I diabetes, rheumatoid arthritis and systemic lupus erythematosus, and patients of these diseases have been shown to have elevated levels of circulating sIL7R that correlate with higher disease activity (Badot et al., 2011; Badot et al., 2013; Lauwerys et al., 2014; Monti et al., 2013). Therefore, the therapeutic SM-ASOs could be used to treat numerous diseases or disorders that have elevated levels of sIL7R.

Cancer is the second leading cause of death in the United States. Effective treatment options for many cancers are lacking given the many etiologies that orchestrate it. Immuno-oncology is a rapidly growing field that uses the body's immune system as novel treatments for previously intractable cancers. Immune check point inhibitors, such as monoclonal antibodies that target the negative immune regulators CTLA-4 (Ipilimumab (Yervoy, Bristol-Myers Squibb)) and PD-1 (Pembrolizumab (Keytruda, Merck) and Nivolumab (Opdivo, Bristol-Myers Squibb)) are FDA approved. An example of their potential is the study in patients with previously untreated, and inoperable or metastatic melanoma who were treated with a combination of Nivolumab and Ipilimumab, which reported a 50% overall response rate (Larkin J, Chiarion-Sileni V, Gonzalez R, Grob J J, Cowey C L, et al. 2015. N Engl J Med 373: 23-34).

Although recent cancer immunotherapies have provided new hope, unfortunately, most patients receiving immunotherapy do not respond well and some cancer types (e.g., hepatocellular carcinoma) have low response rates. For instance, while Nivolumab is FDA-approved for patients with hepatocellular carcinoma who failed to respond to the kinase inhibitor Sorafenib, the overall response rate among 154 patients tested was only 14.3% and a complete response was observed in only 3 patients (NCT01658878). Although encouraging, these data indicate that there is a critical need to increase the level of response level in a given patient and the response rate among a patient population.

The need to increase immunotherapeutic response rate has inspired the intense search for markers (e.g., PD-L1 expression and high microsatellite instability in tumor cells) that predict success for the currently deployed immune check point blockers. Equally exciting, although less developed, is the search for pro-immune modulators that could synergize with check point blockade. Elevated levels of sIL7R are thought to enhance immunological responses by potentiating the bioavailability and/or bioactivity of the cytokine IL7, leading to enhanced survival of T cells (Lundstrom et al., 2013). This creates a pro-inflammatory environment that has the potential to increase immune responses against cancers. Accordingly, the present invention uses SM-ASOs that increase sIL7R, such as lead SM-ASOs IL7R-001 and IL7R-004, and additional SM-ASOs in Table 3 and Table 4, as a novel immunotherapy against cancer.

TABLE 3
Sequence of SM-ASOs that increase sIL7R (pro-sIL7R SM-ASOs).

| | | | | SEQ |
|---|---|---|---|---|
| | | | Disease | ID |
| SM-ASO | SM-ASO Sequence* | Efficiency\hat{ } | application | NO |
| IL7R-001 | CUUUAUUAGUUGAAGAAGGUCACCU | +++ | Immuno-oncology | 14 |
| IL7R-002 | CUCAAAAUGCUGAUG | +++ | Immuno-oncology | 15 |
| IL7R-003 | GACCAACAGAGCGACAGAGAAAAAA | +++ | Immuno-oncology | 16 |
| IL7R-004 | ACAGAGCGACAGAGA | +++ | Immuno-oncology | 17 |
| IL7R-007 | UCCCACACAAUCACC | + | Immuno-oncology | 18 |
| IL7R-013 | CAUCAAUAAAUGGGACUUAAGC | + | Immuno-oncology | 19 |
| IL7R-015 | UGGUUUUCUCAUCAAUAAAUG | +++ | Immuno-oncology | 20 |
| IL7R-018 | UCCCCUUUGUGGUUU | ++ | Immuno-oncology | 21 |
| IL7R-019 | CCUUAAUCCCCUUUGU | +++ | Immuno-oncology | 22 |
| IL7R-020 | UGAAAUGCCUUAAUCCCC | ++ | Immuno-oncology | 23 |
| IL7R-021 | UCGUGAAAUGCCUUAA | + | Immuno-oncology | 24 |
| IL7R-023 | GGCACUAAAUUCGUGA | + | Immuno-oncology | 25 |
| IL7R-031 | UUUACUUAGUAAUGUGGG | + | Immuno-oncology | 26 |
| IL7R-032 | UUAGUAAUGUGGGCC | ++ | Immuno-oncology | 27 |
| IL7R-033 | AAUGUGGACCCACUU | + | Immuno-oncology | 28 |
| IL7R-034 | GGGUCCACUUAUUAU | + | Immuno-oncology | 29 |
| IL7R-039 | UUGCUUUUCAGUUAAGAGA | + | Immuno-oncology | 30 |
| IL7R-042 | UUUCAGUUAAGAGACAUAUUUG | +++ | Immuno-oncology | 31 |
| IL7R-043 | GUUAAGAGACAUAUUUGACA | ++ | Immuno-oncology | 32 |
| IL7R-044 | GAGACAUAUUUGACAGC | + | Immuno-oncology | 33 |
| IL7R-045 | AUAUUUGACAGCUUUAU | + | Immuno-oncology | 34 |
| IL7R-046 | UGACAGCUUUAUGGA | ++ | Immuno-oncology | 35 |
| IL7R-047 | GCUUUACGGAGGGAU | + | Immuno-oncology | 36 |
| IL7R-048 | AUGGAGGGAUUUUGGUU | ++ | Immuno-oncology | 37 |
| IL7R-049 | GGGAUUUUGGUUUAAAAGGCA | +++ | Immuno-oncology | 38 |
| IL7R-050 | UUUGGUUUAAAAGGCAUUGAC | ++ | Immuno-oncology | 39 |
| IL7R-051 | UUUAAAAGGCAUUGACUUGGG | + | Immuno-oncology | 40 |
| IL7R-053 | AUUGACUUGGGUGAC | + | Immuno-oncology | 41 |
| IL7R-054 | CUUGGGUGACCAGGC | + | Immuno-oncology | 42 |
| IL7R-058 | ACUGGGCACUAAAUU | + | Immuno-oncology | 43 |
| IL7R-059 | GGGAUACUGGGCACU | ++ | Immuno-oncology | 44 |
| IL7R-060 | AGAUAGGGAUACUGG | ++ | Immuno-oncology | 45 |
| IL7R-061 | UGAGGAUAGAUAGGGAU | ++ | Immuno-oncology | 46 |
| IL7R-062 | CGCUGAGGAUAGAUA | + | Immuno-oncology | 47 |
| IL7R-063 | GAAAUUCGCUGAGGAU | + | Immuno-oncology | 48 |
| IL7R-064 | UGUGGAAAUUCGCUG | + | Immuno-oncology | 49 |
| IL7R-065 | CUUAUGAAAUUAACUGUGGAAAUU | ++ | Immuno-oncology | 50 |

*Sequences are shown for RNA SM-ASOs; for DNA SM-ASOs U's are replaced with T's. Nucleotides highlighted in bold and underlined indicate positions where a mismatch was engineered to disrupt potential secondary structures that could limit the activity of the SM-ASO.
\hat{ }Efficacy scale: low (+), intermediate (++), and high (+++).

TABLE 4
Target sequence of SM-ASOs that increase sIL7R (pro-sIL7R SM-ASOs).

| | | | | SEQ |
|---|---|---|---|---|
| | | | Disease | ID |
| SM-ASO | Target Sequence* | Efficiency\hat{ } | application | NO |
| IL7R-001 | AGGUGACCUUCUUCAACUAAUAAAG | +++ | Immuno-oncology | 14 |
| IL7R-002 | CAUCAGCAUUUUGAG | +++ | Immuno-oncology | 15 |
| IL7R-003 | UUUUUUCUCUGUCGCUCUGUUGGU | +++ | Immuno-oncology | 16 |
| | C | | | |
| IL7R-004 | UCUCUGUCGCUCUGU | +++ | Immuno-oncology | 17 |
| IL7R-007 | GGUGAUUGUGUGGGA | + | Immuno-oncology | 18 |
| IL7R-013 | GCUUAAGCCCCAUUUAUUGAUG | + | Immuno-oncology | 19 |
| IL7R-015 | CAUUUAUUGAUGAGAAAACCA | +++ | Immuno-oncology | 20 |
| IL7R-018 | AAACCACAAAGGGGA | ++ | Immuno-oncology | 21 |
| IL7R-019 | ACAAAGGGGAUUAAGG | +++ | Immuno-oncology | 22 |
| IL7R-020 | GGGGAUUAAGGCAUUUCA | ++ | Immuno-oncology | 23 |
| IL7R-021 | UUAAGGCAUUUCACGA | + | Immuno-oncology | 24 |
| IL7R-023 | UCACGAAUUUAGUGCC | + | Immuno-oncology | 25 |
| IL7R-031 | CCCACAUUACUAAGUAAA | + | Immuno-oncology | 26 |
| IL7R-032 | GGCCCACAUUACUAA | ++ | Immuno-oncology | 27 |
| IL7R-033 | AAGUGGGCCCACAUU | + | Immuno-oncology | 28 |
| IL7R-034 | AUAAUAAGUGGGCCC | + | Immuno-oncology | 29 |
| IL7R-039 | UCUCUUAACUGAAAAGCAA | + | Immuno-oncology | 30 |
| IL7R-042 | CAAAUAUGUCUCUUAACUGAAA | +++ | Immuno-oncology | 31 |
| IL7R-043 | UGUCAAAUAUGUCUCUUAAC | ++ | Immuno-oncology | 32 |
| IL7R-044 | GCUGUCAAAUAUGUCUC | + | Immuno-oncology | 33 |
| IL7R-045 | AUAAAGCUGUCAAAUAU | + | Immuno-oncology | 34 |
| IL7R-046 | UCCAUAAAGCUGUCA | ++ | Immuno-oncology | 35 |
| IL7R-047 | AUCCCUCCAUAAAGC | + | Immuno-oncology | 36 |
| IL7R-048 | AACCAAAAUCCCUCCAU | ++ | Immuno-oncology | 37 |
| IL7R-049 | UGCCUUUUAAACCAAAAUCCC | +++ | Immuno-oncology | 38 |
| IL7R-050 | GUCAAUGCCUUUUAAACCAAA | ++ | Immuno-oncology | 39 |
| IL7R-051 | CCCAAGUCAAUGCCUUUUAAA | + | Immuno-oncology | 40 |
| IL7R-053 | GUCACCCAAGUCAAU | + | Immuno-oncology | 41 |
| IL7R-054 | GCCUGGUCACCCAAG | + | Immuno-oncology | 42 |
| IL7R-058 | AAUUUAGUGCCCAGU | + | Immuno-oncology | 43 |
| IL7R-059 | AGUGCCCAGUAUCCC | ++ | Immuno-oncology | 44 |
| IL7R-060 | CCAGUAUCCCUAUCU | ++ | Immuno-oncology | 45 |
| IL7R-061 | AUCCCUAUCUAUCCUCA | ++ | Immuno-oncology | 46 |
| IL7R-062 | UAUCUAUCCUCAGCG | + | Immuno-oncology | 47 |
| IL7R-063 | AUCCUCAGCGAAUUUC | + | Immuno-oncology | 48 |
| IL7R-064 | CAGCGAAUUUCCACA | + | Immuno-oncology | 49 |
| IL7R-065 | AAUUUCCACAGUUAAUUUCAUAAG | ++ | Immuno-oncology | 50 |

*Nucleotides highlighted in bold and underlined indicate positions where a mismatch was engineered at the complementary position in the SM-ASO to disrupt potential secondary structures that could limit the activity of the SM-ASO.
\hat{ }Efficacy scale: low (+), intermediate (++), and high (+++).

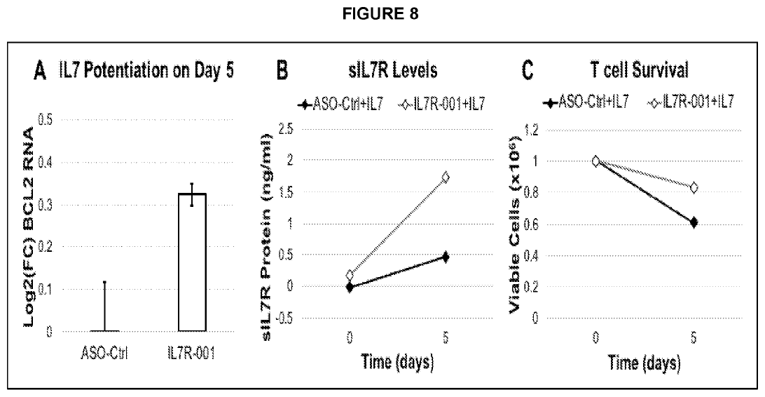

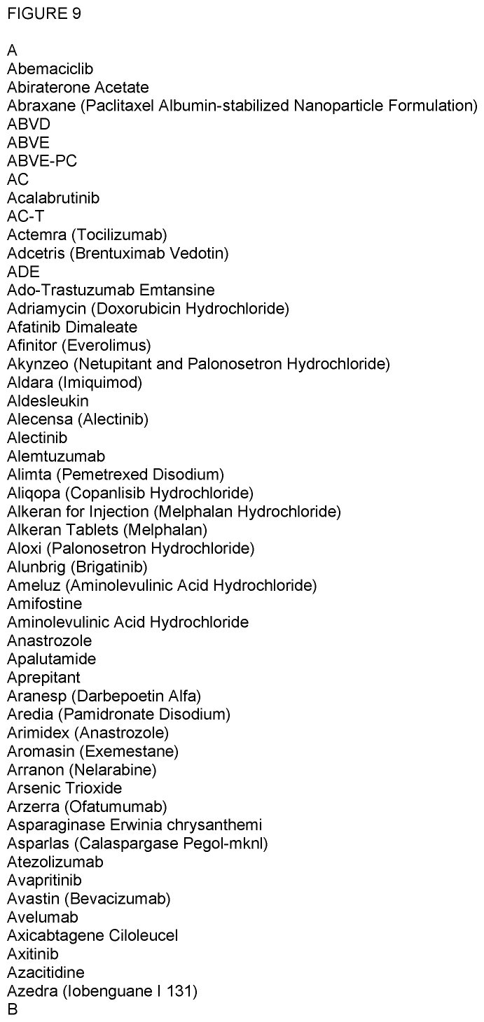

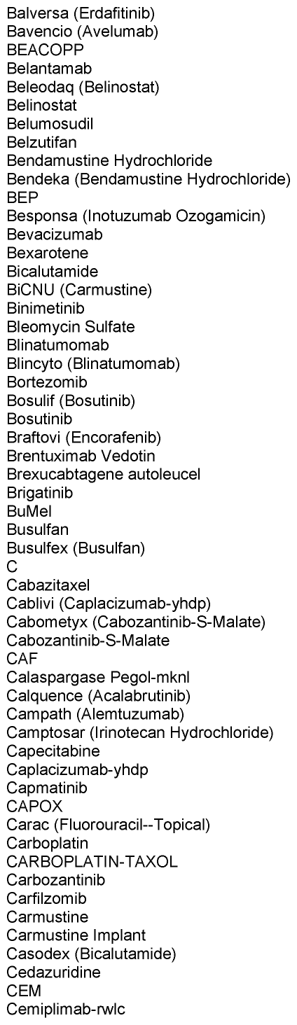

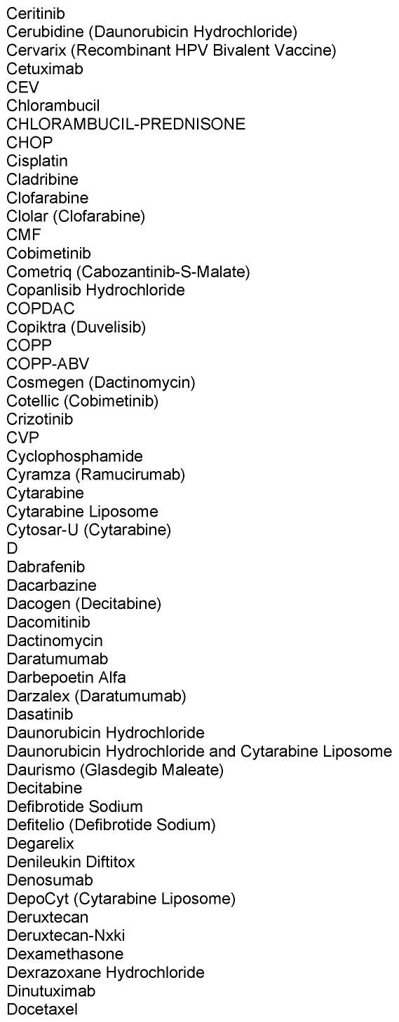

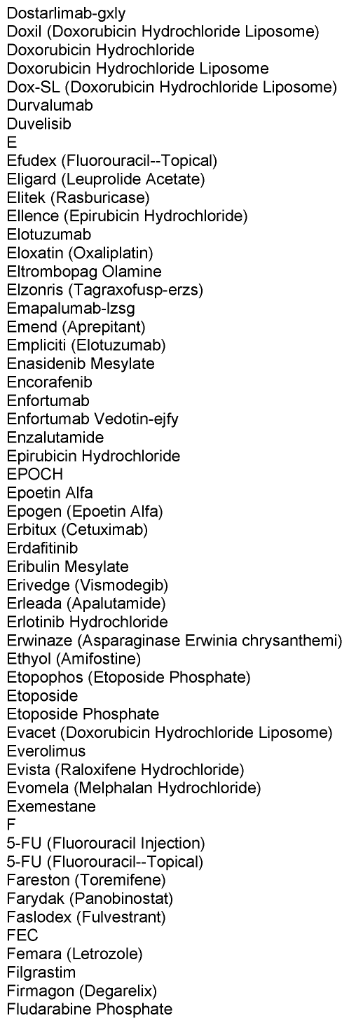

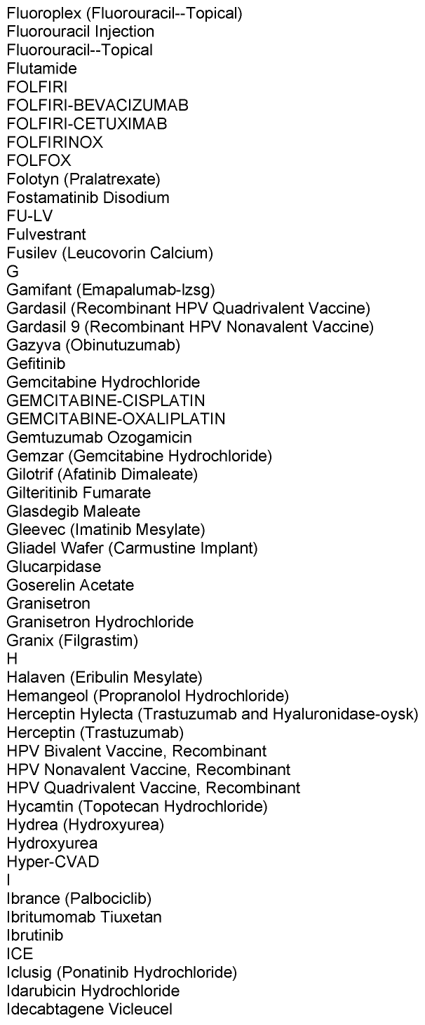

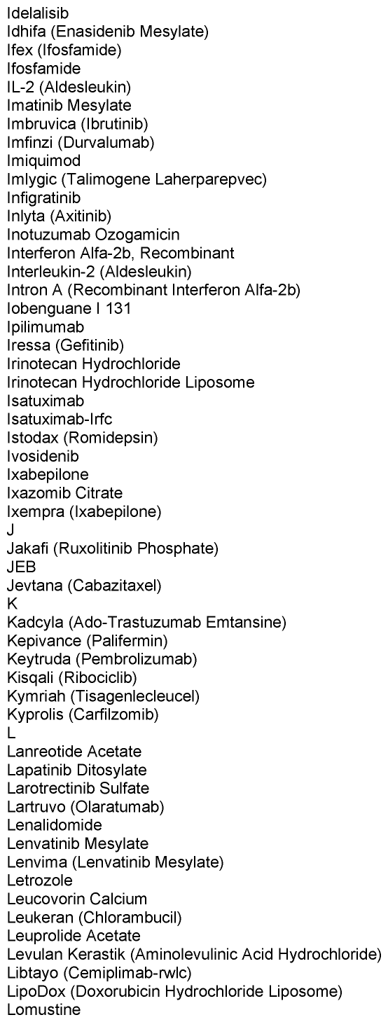

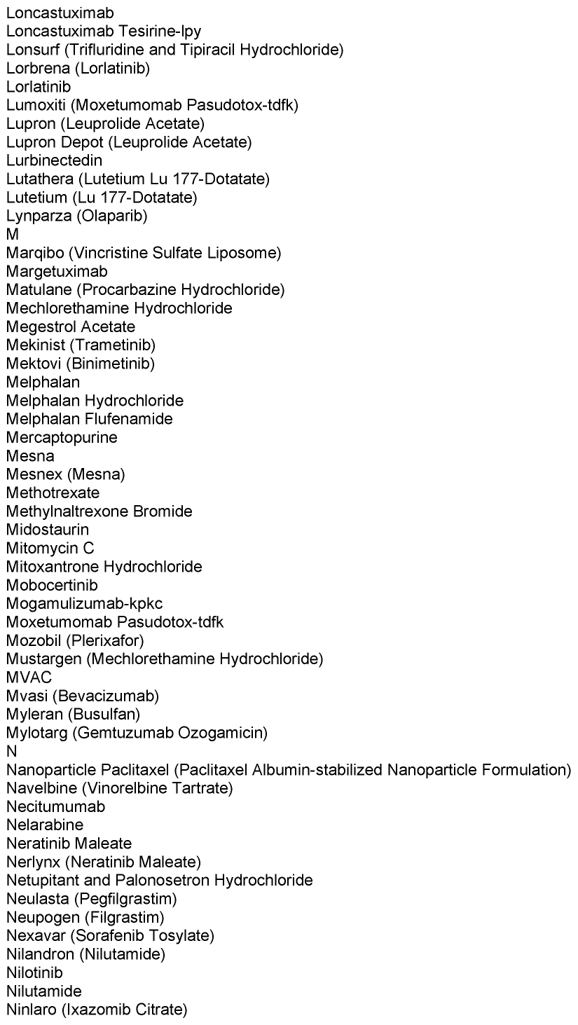

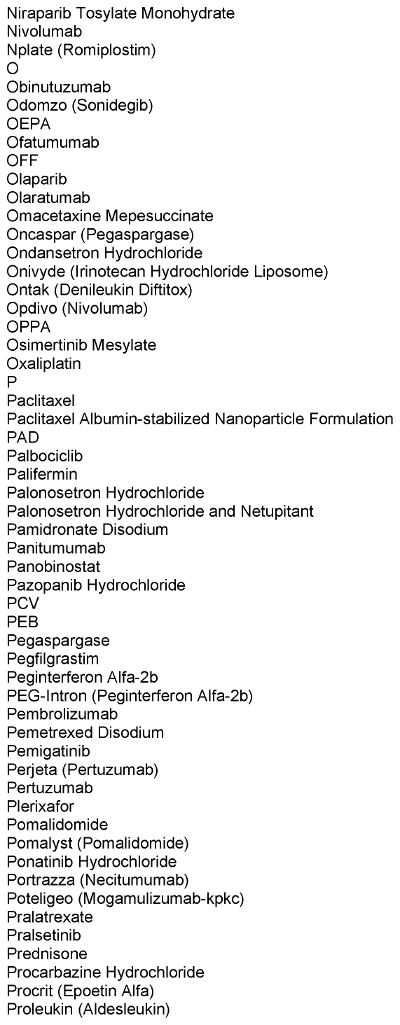

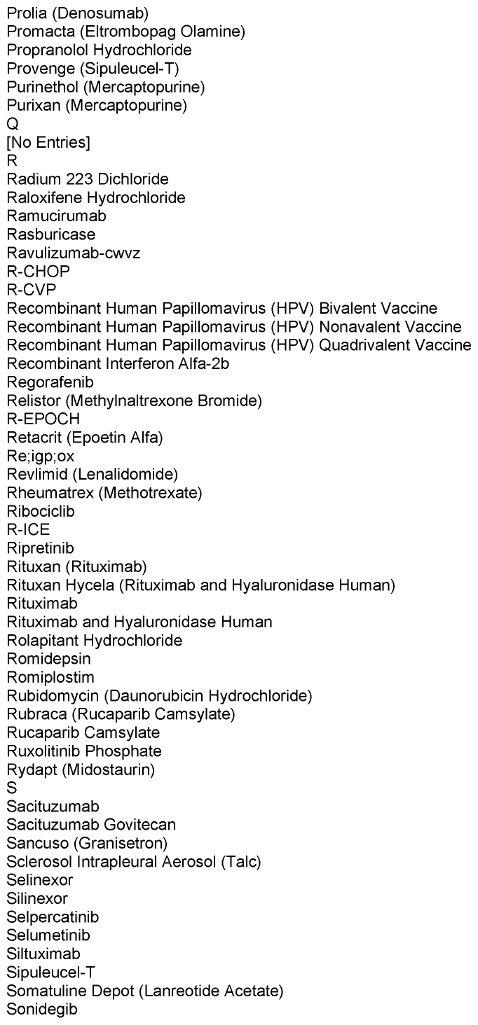

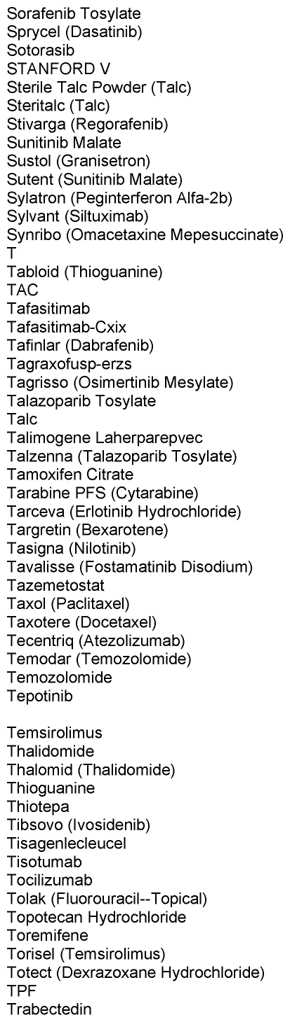

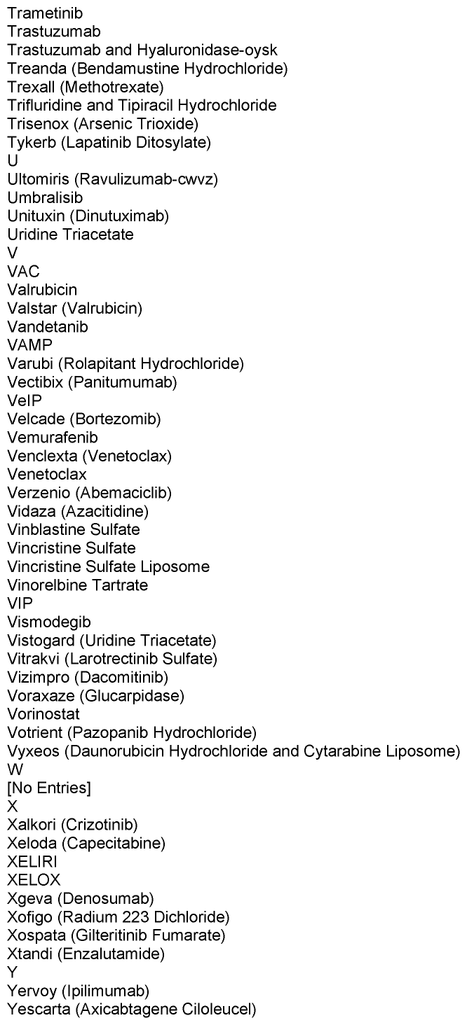

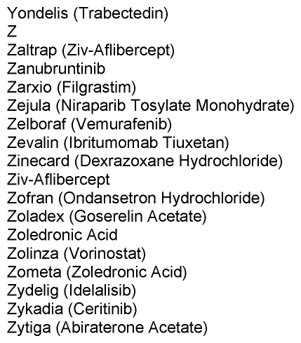

A composition of the present invention comprises an oligonucleotide, including an ASO or SM-ASO. A composition can also comprise one or more additional therapeutics used to treat a cancer. A list of such additional therapeutics is found in FIG. 8. In an embodiment, a composition comprises an oligonucleotide, including an ASO or SM-ASO and an additional therapeutic, including those set forth in FIG. 8.

In other aspects of this embodiment, a composition compound disclosed herein reduces the size of a tumor by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, a composition disclosed herein reduces the size of a tumor from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

A composition disclosed herein is in an amount sufficient to allow customary administration to an individual. In aspects of this embodiment, an ASO or SM-ASO disclosed herein may be, e.g., at least 5 ng, at least 10 ng, at least 15 ng, at least 20 ng, at least 25 ng, at least 30 ng, at least 35 ng, at least 40 ng, at least 45 ng, at least 50 ng, at least 55 ng, at least 60 ng, at least 65 ng, at least 70 ng, at least 75 ng, at least 80 ng, at least 85 ng, at least 90 ng, at least 95 ng, or at least 100 ng, or at least 200 ng, or at least 300 ng, or at least 400 ng, or at least 500 ng, or at least 600 ng, or at least 700 ng, or at least 800 ng, or at least 900 ng, at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg of a composition. In other aspects of this embodiment, an ASO or SM-ASO disclosed herein may be, e.g., at least 5 ng, at least 10 ng, at least 15 ng, at least 20 ng, at least 25 ng, at least 30 ng, at least 35 ng, at least 40 ng, at least 45 ng, at least 50 ng, at least 55 ng, at least 60 ng, at least 65 ng, at least 70 ng, at least 75 ng, at least 80 ng, at least 85 ng, at least 90 ng, at least 95 ng, or at least 100 ng, or at least 200 ng, or at least 300 ng, or at least 400 ng, or at least 500 ng, or at least 600 ng, or at least 700 ng, or at least 800 ng, or at least 900 ng, at least 5 mg, at least 10 mg, at least 20 mg, at least 25 mg, at least 50 mg, at least 75 mg, at least 100 mg, at least 200 mg, at least 300 mg, at least 400 mg, at least 500 mg, at least 600 mg, at least 700 mg, at least 800 mg, at least 900 mg, at least 1,000 mg, at least 1,100 mg, at least 1,200 mg, at least 1,300 mg, at least 1,400 mg, or at least 1,500 mg of a composition. In yet other aspects of this embodiment, an ASO or SM-ASO disclosed herein may be in the range of, e.g., about 5 ng to about 100 ng, about 10 ng to about 100 ng, about 50 ng to about 150 ng, about 100 ng to about 250 ng, about 150 ng to about 350 ng, about 250 ng to about 500 ng, about 350 ng to about 600 ng, about 500 ng to about 750 ng, about 600 ng to about 900 ng, about 750 ng to about 1,000 ng, about 850 ng to about 1,200 ng, or about 1,000 ng to about 1,500 ng, about 5 mg to about 100 mg, about 10 mg to about 100 mg, about 50 mg to about 150 mg, about 100 mg to about 250 mg, about 150 mg to about 350 mg, about 250 mg to about 500 mg, about 350 mg to about 600 mg, about 500 mg to about 750 mg, about 600 mg to about 900 mg, about 750 mg to about 1,000 mg, about 850 mg to about 1,200 mg, or about 1,000 mg to about 1,500 mg.

In still other aspects of this embodiment, an ASO or SM-ASO disclosed herein may be in the range of, e.g., about 10 ng to about 250 ng, about 10 ng to about 500 ng, about 10 ng to about 750 ng, about 10 ng to about 1,000 ng, about 10 ng to about 1,500 ng, about 50 ng to about 250 ng, about 50 ng to about 500 ng, about 50 ng to about 750 ng, about 50 ng to about 1,000 ng, about 50 ng to about 1,500 ng, about 100 ng to about 250 ng, about 100 ng to about 500 ng, about 100 ng to about 750 ng, about 100 ng to about 1,000 ng, about 100 ng to about 1,500 ng, about 200 ng to about 500 ng, about 200 ng to about 750 ng, about 200 ng to about 1,000 ng, about 200 ng to about 1,500 ng, about 5 ng to about 1,500 ng, about 5 ng to about 1,000 ng, or about 5 ng to about 250 ng, 10 mg to about 250 mg, about 10 mg to about 500 mg, about 10 mg to about 750 mg, about 10 mg to about 1,000 mg, about 10 mg to about 1,500 mg, about 50 mg to about 250 mg, about 50 mg to about 500 mg, about 50 mg to about 750 mg, about 50 mg to about 1,000 mg, about 50 mg to about 1,500 mg, about 100 mg to about 250 mg, about 100 mg to about 500 mg, about 100 mg to about 750 mg, about 100 mg to about 1,000 mg, about 100 mg to about 1,500 mg, about 200 mg to about 500 mg, about 200 mg to about 750 mg, about 200 mg to about 1,000 mg, about 200 mg to about 1,500 mg, about 5 mg to about 1,500 mg, about 5 mg to about 1,000 mg, or about 5 mg to about 250 mg.

A composition disclosed herein may comprise a solvent, emulsion or other diluent in an amount sufficient to dissolve a composition disclosed herein. In other aspects of this embodiment, a composition disclosed herein may comprise a solvent, emulsion or a diluent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In other aspects of this embodiment, a composition disclosed herein may comprise a solvent, emulsion or other diluent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

The final concentration of an ASO or SM-ASO disclosed herein in a composition disclosed herein may be of any concentration desired. In an aspect of this embodiment, the final concentration of an ASO or SM-ASO in a composition may be a therapeutically effective amount. In other aspects of this embodiment, the final concentration of an ASO or SM-ASO in a composition may be, e.g., at least 0.00001 mg/mL, at least 0.0001 mg/mL, at least 0.001 mg/mL, at least 0.01 mg/mL, at least 0.1 mg/mL, at least 1 mg/mL, at least 10 mg/mL, at least 25 mg/mL, at least 50 mg/mL, at least 100 mg/mL, at least 200 mg/mL, at least 500 mg/mL, at least 700 mg/mL, at least 1,000 mg/mL, or at least 1,200 mg/mL. In other aspects of this embodiment, the concentration of an ASO or SM-ASO disclosed herein in the solution may be, e.g., at most 1,000 ng/mL, at most 1,100 ng/mL, at most 1,200 ng/mL, at most 1,300 ng/mL, at most 1,400 ng/mL, at most 1,500 ng/mL, at most 2,000 ng/mL, at most 2,000 ng/mL, or at most 3,000 ng/mL, at most 1,000 mg/mL, at most 1,100 mg/mL, at most 1,200 mg/mL, at most 1,300 mg/mL, at most 1,400 mg/mL, at most 1,500 mg/mL, at most 2,000 mg/mL, at most 2,000 mg/mL, or at most 3,000 mg/mL. In other aspects of this embodiment, the final concentration of an ASO or SM-ASO in a composition may be in a range of, e.g., about 0.00001 ng/mL to about 3,000 ng/mL, about 0.0001 ng/mL to about 3,000 ng/mL, about 0.01 ng/mL to about 3,000 ng/mL, about 0.1 ng/mL to about 3,000 ng/mL, about 1 ng/mL to about 3,000 ng/mL, about 250 ng/mL to about 3,000 ng/mL, about 500 ng/mL to about 3,000 ng/mL, about 750 ng/mL to about 3,000 ng/mL, about 1,000 ng/mL to about 3,000 ng/mL, about 100 ng/mL to about 2,000 ng/mL, about 250 ng/mL to about 2,000 ng/mL, about 500 ng/mL to about 2,000 ng/mL, about 750 ng/mL to about 2,000 ng/mL, about 1,000 ng/mL to about 2,000 ng/mL, about 100 ng/mL to about 1,500 ng/mL, about 250 ng/mL to about 1,500 ng/mL, about 500 ng/mL to about 1,500 ng/mL, about 750 ng/mL to about 1,500 ng/mL, about 1,000 ng/mL to about 1,500 ng/mL, about 100 ng/mL to about 1,200 ng/mL, about 250 ng/mL to about 1,200 ng/mL, about 500 ng/mL to about 1,200 ng/mL, about 750 ng/mL to about 1,200 ng/mL, about 1,000 ng/mL to about 1,200 ng/mL, about 100 ng/mL to about 1,000 ng/mL, about 250 ng/mL to about 1,000 ng/mL, about 500 ng/mL to about 1,000 ng/mL, about 750 ng/mL to about 1,000 ng/mL, about 100 ng/mL to about 750 ng/mL, about 250 ng/mL to about 750 ng/mL, about 500 ng/mL to about 750 ng/mL, about 100 ng/mL to about 500 ng/mL, about 250 ng/mL to about 500 ng/mL, about 0.00001 ng/mL to about 0.0001 ng/mL, about 0.00001 ng/mL to about 0.001 ng/mL, about 0.00001 ng/mL to about 0.01 ng/mL, about 0.00001 ng/mL to about 0.1 ng/mL, about 0.00001 ng/mL to about 1 ng/mL, about 0.001 ng/mL to about 0.01 ng/mL, about 0.001 ng/mL to about 0.1 ng/mL, about 0.001 ng/mL to about 1 ng/mL, about 0.001 ng/mL to about 10 ng/mL, or about 0.001 ng/mL to about 100 ng/mL, about 0.00001 mg/mL to about 3,000 mg/mL, about 0.0001 mg/mL to about 3,000 mg/mL, about 0.01 mg/mL to about 3,000 mg/mL, about 0.1 mg/mL to about 3,000 mg/mL, about 1 mg/mL to about 3,000 mg/mL, about 250 mg/mL to about 3,000 mg/mL, about 500 mg/mL to about 3,000 mg/mL, about 750 mg/mL to about 3,000 mg/mL, about 1,000 mg/mL to about 3,000 mg/mL, about 100 mg/mL to about 2,000 mg/mL, about 250 mg/mL to about 2,000 mg/mL, about 500 mg/mL to about 2,000 mg/mL, about 750 mg/mL to about 2,000 mg/mL, about 1,000 mg/mL to about 2,000 mg/mL, about 100 mg/mL to about 1,500 mg/mL, about 250 mg/mL to about 1,500 mg/mL, about 500 mg/mL to about 1,500 mg/mL, about 750 mg/mL to about 1,500 mg/mL, about 1,000 mg/mL to about 1,500 mg/mL, about 100 mg/mL to about 1,200 mg/mL, about 250 mg/mL to about 1,200 mg/mL, about 500 mg/mL to about 1,200 mg/mL, about 750 mg/mL to about 1,200 mg/mL, about 1,000 mg/mL to about 1,200 mg/mL, about 100 mg/mL to about 1,000 mg/mL, about 250 mg/mL to about 1,000 mg/mL, about 500 mg/mL to about 1,000 mg/mL, about 750 mg/mL to about 1,000 mg/mL, about 100 mg/mL to about 750 mg/mL, about 250 mg/mL to about 750 mg/mL, about 500 mg/mL to about 750 mg/mL, about 100 mg/mL to about 500 mg/mL, about 250 mg/mL to about 500 mg/mL, about 0.00001 mg/mL to about 0.0001 mg/mL, about 0.00001 mg/mL to about 0.001 mg/mL, about 0.00001 mg/mL to about 0.01 mg/mL, about 0.00001 mg/mL to about 0.1 mg/mL, about 0.00001 mg/mL to about 1 mg/mL, about 0.001 mg/mL to about 0.01 mg/mL, about 0.001 mg/mL to about 0.1 mg/mL, about 0.001 mg/mL to about 1 mg/mL, about 0.001 mg/mL to about 10 mg/mL, or about 0.001 mg/mL to about 100 mg/mL.

Aspects of the present specification disclose, in part, treating an individual suffering from cancer. As used herein, the term “treating,” refers to reducing or eliminating in an individual a clinical symptom of cancer; or delaying or preventing in an individual the onset of a clinical symptom of cancer. For example, the term “treating” can mean reducing a symptom of a condition characterized by a cancer, including, but not limited to, tumor size, by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual symptoms associated with cancer are well known and can be determined by a person of ordinary skill in the art by taking into account factors, including, without limitation, the location of the cancer, the cause of the cancer, the severity of the cancer, and/or the tissue or organ affected by the cancer. Those of skill in the art will know the appropriate symptoms or indicators associated with a specific type of cancer and will know how to determine if an individual is a candidate for treatment as disclosed herein.

In aspects of this embodiment, a therapeutically effective amount of a composition disclosed herein reduces a symptom associated with cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a composition disclosed herein reduces a symptom associated with cancer by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a composition disclosed herein reduces a symptom associated with cancer by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In yet other aspects of this embodiment, a therapeutically effective amount of an ASO or SM-ASO disclosed herein generally is in the range of about 0.001 mg/kg/day to about 100 mg/kg/day. In aspects of this embodiment, an effective amount of an ASO or SM-ASO disclosed herein may be, e.g., at least 0.001 mg/kg/day, at least 0.01 mg/kg/day, at least 0.1 mg/kg/day, at least 1.0 mg/kg/day, at least 5.0 mg/kg/day, at least 10 mg/kg/day, at least 15 mg/kg/day, at least 20 mg/kg/day, at least 25 mg/kg/day, at least 30 mg/kg/day, at least 35 mg/kg/day, at least 40 mg/kg/day, at least 45 mg/kg/day, or at least 50 mg/kg/day. In other aspects of this embodiment, an effective amount of an ASO or SM-ASO disclosed herein may be in the range of, e.g., about 0.001 mg/kg/day to about 10 mg/kg/day, about 0.001 mg/kg/day to about 15 mg/kg/day, about 0.001 mg/kg/day to about 20 mg/kg/day, about 0.001 mg/kg/day to about 25 mg/kg/day, about 0.001 mg/kg/day to about 30 mg/kg/day, about 0.001 mg/kg/day to about 35 mg/kg/day, about 0.001 mg/kg/day to about 40 mg/kg/day, about 0.001 mg/kg/day to about 45 mg/kg/day, about 0.001 mg/kg/day to about 50 mg/kg/day, about 0.001 mg/kg/day to about 75 mg/kg/day, or about 0.001 mg/kg/day to about 100 mg/kg/day. In yet other aspects of this embodiment, an effective amount of an ASO or SM-ASO disclosed herein may be in the range of, e.g., about 0.01 mg/kg/day to about 10 mg/kg/day, about 0.01 mg/kg/day to about 15 mg/kg/day, about 0.01 mg/kg/day to about 20 mg/kg/day, about 0.01 mg/kg/day to about 25 mg/kg/day, about 0.01 mg/kg/day to about 30 mg/kg/day, about 0.01 mg/kg/day to about 35 mg/kg/day, about 0.01 mg/kg/day to about 40 mg/kg/day, about 0.01 mg/kg/day to about 45 mg/kg/day, about 0.01 mg/kg/day to about 50 mg/kg/day, about 0.01 mg/kg/day to about 75 mg/kg/day, or about 0.01 mg/kg/day to about 100 mg/kg/day. In still other aspects of this embodiment, an effective amount of an ASO or SM-ASO disclosed herein may be in the range of, e.g., about 0.1 mg/kg/day to about 10 mg/kg/day, about 0.1 mg/kg/day to about 15 mg/kg/day, about 0.1 mg/kg/day to about 20 mg/kg/day, about 0.1 mg/kg/day to about 25 mg/kg/day, about 0.1 mg/kg/day to about 30 mg/kg/day, about 0.1 mg/kg/day to about 35 mg/kg/day, about 0.1 mg/kg/day to about 40 mg/kg/day, about 0.1 mg/kg/day to about 45 mg/kg/day, about 0.1 mg/kg/day to about 50 mg/kg/day, about 0.1 mg/kg/day to about 75 mg/kg/day, or about 0.1 mg/kg/day to about 100 mg/kg/day.

In a composition disclosed herein typically may be between about 0.01% to about 45% by weight of an ASO and/or SM-ASO. In aspects of this embodiment, an amount of an ASO and/or SM-ASO disclosed herein may be from, e.g., about 0.1% to about 45% by weight, about 0.1% to about 40% by weight, about 0.1% to about 35% by weight, about 0.1% to about 30% by weight, about 0.1% to about 25% by weight, about 0.1% to about 20% by weight, about 0.1% to about 15% by weight, about 0.1% to about 10% by weight, about 0.1% to about 5% by weight, about 1% to about 45% by weight, about 1% to about 40% by weight, about 1% to about 35% by weight, about 1% to about 30% by weight, about 1% to about 25% by weight, about 1% to about 20% by weight, about 1% to about 15% by weight, about 1% to about 10% by weight, about 1% to about 5% by weight, about 5% to about 45% by weight, about 5% to about 40% by weight, about 5% to about 35% by weight, about 5% to about 30% by weight, about 5% to about 25% by weight, about 5% to about 20% by weight, about 5% to about 15% by weight, about 5% to about 10% by weight, about 10% to about 45% by weight, about 10% to about 40% by weight, about 10% to about 35% by weight, about 10% to about 30% by weight, about 10% to about 25% by weight, about 10% to about 20% by weight, about 10% to about 15% by weight, about 15% to about 45% by weight, about 15% to about 40% by weight, about 15% to about 35% by weight, about 15% to about 30% by weight, about 15% to about 25% by weight, about 15% to about 20% by weight, about 20% to about 45% by weight, about 20% to about 40% by weight, about 20% to about 35% by weight, about 20% to about 30% by weight, about 20% to about 25% by weight, about 25% to about 45% by weight, about 25% to about 40% by weight, about 25% to about 35% by weight, or about 25% to about 30% by weight.

A composition or an ASO or SM-ASO is administered to an individual. An individual is typically a human being, but can be an animal, including, but not limited to, dogs, cats, birds, cattle, horses, sheep, goats, reptiles, monkeys and other animals, whether domesticated or not. Typically, any individual who is a candidate for treatment is a candidate with some form of cancer, whether the cancer is benign or malignant, a tumor, solid or otherwise, a cancer cell not located in a tumor or some other form of cancer.

Dosing of a composition to treat a cancer can be single dosage or cumulative (serial dosing), and can be readily determined by one skilled in the art. For instance, treatment of a cancer may comprise a one-time administration of an effective dose of a composition disclosed herein. Alternatively, treatment of a cancer may comprise multiple administrations of an effective dose of a composition carried out over a range of time periods, such as, e.g., once daily, twice daily, trice daily, once every few days, once weekly, once monthly or others. The timing of administration can vary from individual to individual, depending upon such factors as the severity of an individual's symptoms. For example, an effective dose of a composition disclosed herein to treat a cancer can be administered to an individual once daily for an indefinite period of time, or until the individual no longer requires therapy. A person of ordinary skill in the art will recognize that the condition of the individual can be monitored throughout the course of treatment for the cancer and that the effective amount of a composition disclosed herein that is administered can be adjusted accordingly.

In one embodiment, an ASO or SM-ASO disclosed herein is capable of reducing the number of cancer cells or tumor size in an individual suffering from a cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, an ASO or SM-ASO is capable of reducing the number of cancer cells or tumor size in an individual suffering from a cancer by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In a further embodiment, an ASO or SM-ASO and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of an ASO or SM-ASO is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration of an ASO or SM-ASO is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, a therapeutically effective amount of an ASO or SM-ASO disclosed herein reduces or maintains a cancer cell population and/or tumor cell size in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of an ASO or SM-ASO disclosed herein reduces or maintains a cancer cell population and/or tumor cell size in an individual by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of an ASO or SM-ASO disclosed herein reduces or maintains a cancer cell population and/or tumor cell size in an individual by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

A composition comprising an ASO or SM-ASO is administered to an individual suffering from a cancer. An individual suffering from a cancer is typically a human being, but can be an animal, including, but not limited to, dogs, cats, birds, cattle, horses, sheep, goats, reptiles, monkeys and other animals, whether domesticated or not. Typically, any individual who is a candidate for treatment is a candidate with some form of cancer, whether the cancer is benign or malignant, a tumor, solid or otherwise, a cancer cell not located in a tumor or some other form of cancer. Among the most common types of cancer include, but are not limited to, Lymphoma (including Hodgkin lymphoma (HL), non-Hodgkin lymphoma (NHL), and all subtypes), Primary mediastinal large B-cell lymphoma, Leukemia (including acute myeloid leukemia (AML), chronic myeloid leukemia (CML), acute lymphocytic leukemia (ALL), chronic lymphocytic leukemia (CLL), T cell leukemia and all subtypes, B cell leukemia and all subtypes), Colorectal cancer, Liver cancer (including Hepatocellular carcinoma (HCC), Cholangiocarcinoma (bile duct cancer), and Hepatoblastoma), Hepatocellular carcinoma (HCC), Melanoma, Lung cancer (including Small cell lung cancer (SCLC), and Non-small cell lung cancer (NSCLC), Adenocarcinoma, Squamous cell (epidermoid) carcinoma, and Large cell (undifferentiated) carcinoma, Non-small cell lung cancer (NSCLC) (squamous or nonsquamous), Small cell lung cancer, Kidney cancer, Renal cell carcinoma, Squamous cell carcinoma of the esophagus, Head and neck cancer (including squamous or nonsquamous), Squamous cell carcinoma of the head and neck, Urothelial carcinoma, Cervical cancer, Cutaneous squamous cell carcinoma, Endometrial carcinoma, Gastric (stomach) cancer or gastroesophageal junction cancer, Esophageal cancer (including squamous cell carcinoma, and adenocarcinoma), Merkel cell carcinoma, Microsatellite instability-high (MSI-H) or mismatch repair deficient (dMMR) cancer, Solid tumors (including tumor mutational burden-high (TMB-H), or metastatic cancer), Colorectal cancer (including metastatic microsatellite instability-high (MSI-H) or mismatch repair deficient (dMMR) cancer), Triple-negative breast cancer, Triple-positive breast cancer, Brain cancer (including Astrocytoma, Ependymoma, Glioma, Meningioma, Medulloblastoma, Neuroblastoma), Bladder cancer, Childhood cancer (including acute lymphocytic leukemia, brain cancer, neuroblastoma, non-Hodgkin lymphoma, Hodgkin lymphoma, thyroid carcinoma, testicular germ cell tumors), Multiple Myeloma, Ovarian cancer, Pancreatic cancer, Prostate cancer, Sarcoma (including osteosarcoma, chondrosarcoma, liposarcoma, and leiomyosarcoma), Skin cancer (including Basal cell carcinoma (BCC), Squamous cell carcinoma (SCC), Melanoma, Merkel cell carcinoma (MCC), and Kaposi's sarcoma (KS)), Stomach cancer, and Uterine (Endometrial) cancer. Pre-operative evaluation typically includes routine history and physical examination in addition to thorough informed consent disclosing all relevant risks and benefits of the procedure.

A composition disclosed herein may comprise an ASO and/or SM-ASO in a therapeutically effective amount. As used herein, the term “effective amount” is synonymous with “therapeutically effective amount”, “effective dose”, or “therapeutically effective dose” and when used in reference to reducing or maintaining a cancer cell population and/or tumor cell size in an individual refers to the minimum dose of a composition comprising an ASO and/or SM-ASO disclosed herein necessary to achieve the desired therapeutic effect and includes a dose sufficient to reduce or maintain of cancer cell population and/or tumor cell size in an individual. The effectiveness of a composition comprising an ASO and/or SM-ASO disclosed herein capable of reducing or maintaining a cancer cell population and/or tumor cell size in an individual can be determined by observing an improvement in an individual based upon one or more clinical symptoms, and/or physiological indicators associated with reducing or maintaining a cancer cell population and/or tumor cell size in an individual. Maintenance or a reduction of cancer cell population and/or tumor cell size can be indicated by a reduced need for a concurrent therapy. The effectiveness of a composition comprising an ASO and/or SM-ASO disclosed herein capable of reducing or maintaining a cancer cell population and/or tumor cell size in an individual can be determined by observing an improvement in an individual based upon one or more clinical symptoms, and/or physiological indicators associated with a reduction or maintenance of cancer cell population and/or tumor cell size. The effectiveness of a composition comprising an ASO and/or SM-ASO disclosed herein is also capable of prolonging the life of an individual as compared to the same individual if the composition comprising an ASO and/or SM-ASO is not administered. The effectiveness of a composition comprising an ASO and/or SM-ASO disclosed herein is also capable of enhancing the quality of life of an individual as compared to the same individual if the composition comprising an ASO and/or SM-ASO is not administered.

The appropriate effective amount of a composition comprising an ASO and/or SM-ASO disclosed herein to be administered to reduce or maintain of a cancer cell population and/or tumor cell size in an individual condition can be determined by a person of ordinary skill in the art by taking into account factors, including, without limitation, the measured number of cancer cells in blood samples or biopsies or CAT scans, PET scans, NMR and/or sonograms taken from or of the individual, the particular characteristics, history and risk factors of the patient, such as, e.g., age, weight, general health and the like, or any combination thereof. Additionally, where repeated administration of a composition comprising an ASO and/or SM-ASO is used, an effective amount of a composition comprising an ASO and/or SM-ASO will further depend upon factors, including, without limitation, the frequency of administration, the half-life of the cancer therapeutic, or any combination thereof. In is known by a person of ordinary skill in the art that an effective amount of a composition comprising an ASO and/or SM-ASO disclosed herein can be extrapolated from in vitro assays and in vivo administration studies using animal models prior to administration to humans or animals. In an embodiment, a composition comprising an ASO and/or SM-ASO includes an ASO and/or SM-ASO alone or with one or more excipients and/or other active therapeutic.

In an embodiment, in instances in which a composition is administered, without limitation, as individual or separate dosage forms (e.g., capsules or tablets), the kit comprises, without limitation, the composition makes up the composition of the invention, along with instructions for use. In an additional embodiment, a composition, without limitation, may be packaged in any manner suitable for administration, so long as the packaging, when considered along with the instructions for administration, without limitation, clearly indicates the manner in which each of components of the composition to be administered. In a further embodiment, a composition may, without limitation, be combined into a single administrable dosage form such as a capsule, tablet, or other solid or liquid formulation. A composition can be provided to an individual in a package. The package can be a container, for instance, without limitation, a bottle, a canister, a tube or other enclosed vessel. The package can also be a packet, such as a blister pack.

In an embodiment, the composition comprising a therapeutic, may be formulated for either local or systemic delivery using topical, enteral or parenteral routes of administration. Additionally, an ASO or SM-ASO that is disclosed herein may be formulated by itself in a composition or in a composition where it is formulated together with one or more other therapeutics to create a single composition. In a further aspect, a composition comprising an ASO and/or SM-ASO is administered through an intravenous infusion, direct injection into the tumor microenvironment, a combination of both, orally, intrarectally, subcutaneously, intravaginally, intramuscularly, intrathecally, or other commonly used routes of delivery.

An ASO and/or SM-ASO therapeutic disclosed herein, or a composition comprising such a therapeutic, may be made into an inhaled formulation. Inhaled formulations suitable for enteral or parenteral administration include, without limitation, aerosols, dry powders. An ASO and/or SM-ASO therapeutic or composition disclosed herein intended for such administration may be prepared according to any method known to the art for the manufacture of compositions.

In such inhaled dosage forms, the ASO and/or SM-ASO therapeutic or a composition may be prepared for delivery as an aerosol in a liquid propellant for use in a pressurized (PDI) or other metered dose inhaler (MDI). Propellants suitable for use in a PDI or MDI include, without limitation, CFC-12, HFA-134a, HFA-227, HCFC-22 (difluorochloromethane), HFA-152 (difluoroethane and isobutane). An ASO and/or SM-ASO therapeutic may also be delivered using a nebulizers or other aerosol delivery system. An ASO and/or SM-ASO therapeutic may be prepared for delivery as a dry powder for use in a dry powder inhaler (DPI). A dry powder for use in the inhalers will usually have a mass median aerodynamic diameter of less than 30 μm, preferably less than 20 μm and more preferably less than 10 pm. Microparticles having aerodynamic diameters in the range of about 5 μm to about 0.5 pm will generally be deposited in the respiratory bronchioles, whereas smaller particles, having aerodynamic diameters in the range of about 2 μm to about 0.05 pm, are likely to be deposited in the alveoli. A DPI may be a passive delivery mechanism, which relies on the individual's inspiration to introduce the particles into the lungs, or an active delivery mechanism, requiring a mechanism for delivering the powder to the individual. In inhalatory formulations, a therapeutically effective amount of an ASO and/or SM-ASO therapeutic disclosed herein for an inhaled formulation may be between about 0.0001% (w/v) to about 60% (w/v), about 0.001% (w/v) to about 40.0% (w/v), or about 0.01% (w/v) to about 20.0% (w/v). In inhalatory formulations, a therapeutically effective amount of a cancer therapeutic disclosed herein for an inhaled formulation may also be between about 0.0001% (w/w) to about 60% (w/w), about 0.001% (w/w) to about 40.0% (w/w), or about 0.01% (w/w) to about 20.0% (w/w).

An ASO and/or SM-ASO therapeutic disclosed herein, or a composition comprising such an ASO and/or SM-ASO therapeutic, may be made into a solid formulation. Solid formulations suitable for enteral or parenteral administration include, without limitation, capsules, tablets, pills, troches, lozenges, powders and granules suitable for inhalation or for reconstitution into sterile injectable solutions or dispersions. An ASO and/or SM-ASO therapeutic or composition disclosed herein intended for such administration may be prepared according to any method known to the art for the manufacture of pharmaceutical compositions. In such solid dosage forms, the ASO and/or SM-ASO may be admixed with (a) at least one inert customary excipient (or carrier), such as, e.g., sodium citrate or dicalcium phosphate, or (b) fillers or extenders, as for example, starch, lactose, sucrose, glucose, mannitol, isomalt, and silicic acid, (c) binders, such as, e.g., carboxymethylcellulose, alignates, gelatin, polyvinylpyrrolidone, sucrose and acacia, (d) humectants, such as, e.g., glycerol, (e) disintegrating agents, such as, e.g., agar-agar, calcium carbonate, corn starch, potato starch, tapioca starch, alginic acid, certain complex silicates and sodium carbonate, (f) solution retarders, such as, e.g., paraffin, (g) absorption accelerators, such as, e.g., quaternary ammonium compounds, (h) wetting agents, such as, e.g., cetyl alcohol and glycerol monostearate, (i) adsorbents, such as, e.g., kaolin and bentonite, (j) lubricants, such as, e.g., talc, stearic acid, calcium stearate, magnesium stearate, solid polyethylene glycols, sodium lauryl sulfate or mixtures thereof, and (k) buffering agents. The tablets may be uncoated, or they may be coated by known techniques to delay disintegration and absorption in the gastrointestinal tract and thereby provide a sustained action over a longer period. For example, a time delay material such as glyceryl monostearate or glyceryl distearate may be employed. In solid formulations, a therapeutically effective amount of an ASO and/or SM-ASO disclosed herein typically may be between about 0.0001% (w/w) to about 60% (w/w), about 0.001% (w/w) to about 40.0% (w/w), or about 0.01% (w/w) to about 20.0% (w/w).

An ASO and/or SM-ASO disclosed herein, or a composition comprising such an ASO and/or SM-ASO, may be made into a semi-solid formulation. Semi-solid formulations suitable for topical administration include, without limitation, ointments, creams, salves, and gels. An ASO and/or SM-ASO or composition disclosed herein intended for such administration may be prepared according to any method known to the art for the manufacture of pharmaceutical compositions. In semi-solid formulations, a therapeutically effective amount of an ASO and/or SM-ASO disclosed herein typically may be between about 0.0001% (w/v) to about 60% (w/v), about 0.001% (w/v) to about 40.0% (w/v), or about 0.01% (w/v) to about 20.0% (w/v). In semi-solid formulations, a therapeutically effective amount of an ASO and/or SM-ASO disclosed herein typically may also be between about 0.0001% (w/w) to about 60% (w/w), about 0.001% (w/w) to about 40.0% (w/w), or about 0.01% (w/w) to about 20.0% (w/w).disclosed herein, or a composition comprising such an ASO and/or SM-ASO, may be made into a semi-solid formulation. Semi-solid formulations suitable for topical administration include, without limitation, ointments, creams, salves, and gels. An ASO and/or SM-ASO or composition disclosed herein intended for such administration may be prepared according to any method known to the art for the manufacture of compositions. In semi-solid formulations, a therapeutically effective amount of an ASO and/or SM-ASO disclosed herein typically may be between about 0.0001% (w/v) to about 60% (w/v), about 0.001% (w/v) to about 40.0% (w/v), or about 0.01% (w/v) to about 20.0% (w/v). In semi-solid formulations, a therapeutically effective amount of an ASO and/or SM-ASO disclosed herein typically may also be between about 0.0001% (w/w) to about 60% (w/w), about 0.001% (w/w) to about 40.0% (w/w), or about 0.01% (w/w) to about 20.0% (w/w).

An ASO and/or SM-ASO disclosed herein, or a composition comprising such an ASO and/or SM-ASO, may be made into a liquid formulation. Liquid formulations suitable for enteral or parenteral administration include, without limitation, solutions, syrups, elixirs, dispersions, emulsions, and suspensions, including, but not limited, to those used for intravenous administration. An ASO and/or SM-ASO or composition disclosed herein intended for such administration may be prepared according to any method known to the art for the manufacture of compositions. In such liquid dosage forms, an ASO and/or SM-ASO or composition disclosed herein may be admixed with (a) suitable aqueous and nonaqueous carriers, (b) diluents, (c) solvents, such as, e.g., water, ethanol, propylene glycol, polyethylene glycol, glycerol, vegetable oils, such as, e.g., rapeseed oil and olive oil, and injectable organic esters such as ethyl oleate; and/or fluidity agents, such as, e.g., surfactants or coating agents like lecithin. In the case of dispersions and suspensions, fluidity can also be controlled by maintaining a particular particle size. In liquid formulations, a therapeutically effective amount of an ASO and/or SM-ASO disclosed herein typically may be between about 0.0001% (w/v) to about 60% (w/v), about 0.001% (w/v) to about 40.0% (w/v), or about 0.01% (w/v) to about 20.0% (w/v).

Liquid suspensions may be formulated by suspending an ASO and/or SM-ASO disclosed herein in admixture with excipients suitable for the manufacture of aqueous suspensions. Such excipients are suspending agents, for example sodium carboxymethylcellulose, methylcellulose, hydroxypropyl methylcellulose, sodium alginate, pectin, polyvinyl pyrrolidone, polyvinyl alcohol, natural gum, agar, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long-chain aliphatic alcohols, for example heptadecaethyleneoxycetanol, or condensation products of ethylene oxide with partial esters derived from fatty acids, for example polyoxyethylene sorbitan monooleate.

Oily suspensions may be formulated by suspending an ASO and/or SM-ASO disclosed herein in admixture with (a) vegetable oils, such as, e.g., almond oil, arachis oil, avocado oil, canola oil, castor oil, coconut oil, corn oil, cottonseed oil, grape seed oil, hazelnut oil, hemp oil, linseed oil, olive oil, palm oil, peanut oil, rapeseed oil, rice bran oil, safflower oil, sesame oil, soybean oil, soya oil, sunflower oil, walnut oil, wheat germ oil, or a combination thereof, (b) a saturated fatty acid, an unsaturated fatty acid, or a combination thereof, such as, e.g., palmitic acid, stearic acid, oleic acid, linoleic acid, linolenic acid, or a combination thereof, (c) mineral oil such as, e.g., liquid paraffin, (d) surfactants or detergents. The oily suspensions may contain a thickening agent, for example beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the ASO and/or SM-ASO in admixture with a dispersing or wetting agent, suspending agent and one or more preservatives.

An ASO and/or SM-ASO disclosed herein may be in the form of oil-in-water emulsions. The oily phase may be a vegetable oil as disclosed herein or a mineral oil as disclosed herein or mixtures thereof. Suitable emulsifying agents may be naturally occurring gums, such as, e.g., gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate.

An ASO and/or SM-ASO disclosed herein, or a composition comprising such an ASO and/or SM-ASO, may also be incorporated into a composition delivery platform in order to achieve a controlled release profile over time. Such a composition delivery platform comprises an ASO and/or SM-ASO disclosed herein dispersed within a polymer matrix, typically a biodegradable, bioerodible, and/or bioresorbable polymer matrix. As used herein, the term “polymer” refers to synthetic homo- or copolymers, naturally occurring homo- or copolymers, as well as synthetic modifications or derivatives thereof having a linear, branched or star structure. Copolymers can be arranged in any form, such as, e.g., random, block, segmented, tapered blocks, graft, or triblock. Polymers are generally condensation polymers. Polymers can be further modified to enhance their mechanical or degradation properties by introducing cross-linking agents or changing the hydrophobicity of the side residues. If crosslinked, polymers are usually less than 5% crosslinked, usually less than 1% crosslinked.

Suitable polymers include, without limitation, alginates, aliphatic polyesters, polyalkylene oxalates, polyamides, polyamidoesters, polyanhydrides, polycarbonates, polyesters, polyethylene glycol, polyhydroxyaliphatic carboxylic acids, polyorthoesters, polyoxaesters, polypeptides, polyphosphazenes, polysaccharides, and polyurethanes. The polymer usually comprises at least about 10% (w/w), at least about 20% (w/w), at least about 30% (w/w), at least about 40% (w/w), at least about 50% (w/w), at least about 60% (w/w), at least about 70% (w/w), at least about 80% (w/w), or at least about 90% (w/w) of the composition platform. Examples of biodegradable, bioerodible, and/or bioresorbable polymers and methods useful to make a cancer therapeutic delivery platform are described in, e.g., Drost, et. al., Controlled Release Formulation, U.S. Pat. No. 4,756,911; Smith, et. al., Sustained Release Drug Delivery Devices, U.S. Pat. No. 5,378,475; Wong and Kochinke, Formulation for Controlled Release of Drugs by Combining Hydrophilic and Hydrophobic Agents, U.S. Pat. No. 7,048,946; Hughes, et. al., Compositions and Methods for Localized Therapy of the Eye, U.S. Patent Publication 2005/0181017; Hughes, Hypotensive Lipid-Containing Biodegradable Intraocular Implants and Related Methods, U.S. Patent Publication 2005/0244464; Altman, et al., Silk Fibroin Hydrogels and Uses Thereof, U.S. Patent Publication 2011/0008437; each of which is incorporated by reference in its entirety.

A composition delivery platform includes both a sustained release composition delivery platform and an extended-release composition delivery platform. As used herein, the term “sustained release” refers to the release of an ASO and/or SM-ASO disclosed herein over a period of about seven days or more. As used herein, the term “extended release” refers to the release of an ASO and/or SM-ASO disclosed herein over a period of time of less than about seven days.

In aspects of this embodiment, a sustained release composition delivery platform releases an ASO and/or SM-ASO disclosed herein with substantially zero order release kinetics over a period of, e.g., about 7 days after administration, about 15 days after administration, about 30 days after administration, about 45 days after administration, about 60 days after administration, about 75 days after administration, or about 90 days after administration. In other aspects of this embodiment, a sustained release composition delivery platform releases an ASO and/or SM-ASO disclosed herein with substantially zero order release kinetics over a period of, e.g., at least 7 days after administration, at least 15 days after administration, at least 30 days after administration, at least 45 days after administration, at least 60 days after administration, at least 75 days after administration, or at least 90 days after administration.

Various routes of administration can be useful for administering a composition disclosed herein, according to a method for reducing and/or maintaining a cancer cell population and/or tumor cell size in an individual. A composition may be administered to an individual by any of a variety of means depending, e.g., on the specific composition used, or other compound to be included in the composition, and the history, risk factors and symptoms of the individual. As such, topical, enteral, oral, intravenous, subcutaneous, intranasal, rectal, vaginal, intrathecal, intramuscular or parenteral routes of administration may be suitable for reducing or maintaining a cancer cell population and/or tumor cell size in an individual as disclosed herein and such routes include both local and systemic delivery of a cancer therapeutic or composition disclosed herein. Compositions comprising either a single ASO 15 and/or SM-ASO disclosed herein, or two or more ASO's and/or SM-ASO's disclosed herein are intended for inhaled, topical, intranasal, oral, subcutaneous, sublingual, intravenous, intrathecal, rectal and/or vaginal use may be prepared according to any method known to the art for the manufacture of pharmaceutical compositions. A composition disclosed herein can be administered to an individual in a single formulation or in separate formulations, for combined, simultaneous or sequential administration.

Aspects of the present specification disclose, in part, a pharmaceutically acceptable solvent. A solvent is a liquid, solid, or gas that dissolves another solid, liquid, or gaseous (the solute), resulting in a solution. Solvents useful in the compositions disclosed herein include, without limitation, a pharmaceutically acceptable polar aprotic solvent, a pharmaceutically acceptable polar protic solvent and a pharmaceutically acceptable non-polar solvent. A pharmaceutically acceptable polar aprotic solvent includes, without limitation, dichloromethane (DCM), tetrahydrofuran (THF), ethyl acetate, acetone, dimethylformamide (DMF), acetonitrile (MeCN), dimethyl sulfoxide (DMSO). A pharmaceutically acceptable polar protic solvent includes, without limitation, acetic acid, formic acid, ethanol, n-butanol, 1-butanol, 2-butanol, isobutanol, sec-butanol, tert-butanol, n-propanol, isopropanol, 1,2 propan-diol, methanol, glycerol, and water. A pharmaceutically acceptable non-polar solvent includes, without limitation, pentane, cyclopentane, hexane, cyclohexane, benzene, toluene, 1,4-Dioxane, chloroform, n-methyl-pyrrilidone (NMP), and diethyl ether.

Thus, the present inventors have described compositions and methods for using antisense oligonucleotides to control alternative splicing of exon 6 of the Interleukin 7 receptor (IL7R) RNAs for therapeutic intervention in autoimmunity and cancer. These antisense oligonucleotides control splicing of IL7R exon 6 by blocking specific signals embedded in IL7R RNAs. These signals are specific sequences that determine the splicing outcome of IL7R exon 6. Further, specific sequences in IL7R RNAs to be blocked by antisense oligonucleotides to reduce sIL7R are listed in Table 2, including variations of these sequences, any portion of these sequences, or any nucleotides flanking these sequences that increase inclusion of IL7R exon 6, thus decreasing sIL7R secretion. Further, additional signals in IL7R RNAs to be blocked by antisense oligonucleotides to increase sIL7R are listed in Table 4, including variations of these sequences, any portion of these sequences, or any nucleotides flanking these sequences that decrease inclusion of IL7R exon 6, thus increasing sIL7R secretion. Blocking just a few nucleotides of these sequences can affect splicing of exon 6 because the actual element(s) that drives exclusion/inclusion is not the entire targeted sequence but usually a sequence of 4-8 nt within the targeted sequence. For example, an important sequence within the IL7R-005 target sequence is the last 5 nt UGGUC, thus, an ASO blocking just this 5 nt sequence or a few nt of this sequence might be sufficient to cause the desired effect. However, in order to maximize targeting specificity and efficiency for the functional sequence the oligonucleotide is often made to a longer complementary sequence. Finally, it is well known that it is possible to replace one or more bases in the antisense oligonucleotide to modify base-pairing while retaining high affinity, selectivity and efficient antisense activity for said target sequences. Depending on the length of the SM-ASO, non-limiting examples are those having 15, 20, or 25 nucleotides, which may have 70, 75, 80, 84, 85, 87, 88, 90, 92, 93, 94, 95, or 96, percent complementarity and or identity to any of the TARGET SEQ IDs in Tables 2 and 4, or portions thereof, either alone or in combination, fully or partially, or any biologically active permutation of these TARGET SEQ IDs for the treatment of autoimmune diseases, inflammatory diseases or cancer, respectively, or sequences complementary thereto, for use as a composition and in methods for reducing the expression of soluble IL7R by enhancing the inclusion of IL7R exon 6 for treatment of autoimmune and/or inflammatory diseases, or for enhancing the expression of soluble IL7R by reducing the inclusion of IL7R exon 6 for treatment of cancer. For a SM-ASO having 5, 10, 15, 20 or 25 nucleotides the mismatch may be, e.g., 1, 2, 3, 4, 5 or more mismatches.

TABLE 5
Summary of percent complementarity and or identity
(% complementarity and or identity) with up to 8 mismatches for each
of SM-ASOs IL7R-005, IL7R-006, IL7R-001 and IL7R-004.

| | IL7R-005 | IL7R-006 | IL7R-001 | IL7R-004 |
|---|---|---|---|---|
| | (15 nt) | (20 nt) | (25 nt) | (15 nt) |
| | % | % | % | % |
| | com- | com- | com- | com- |
| | plementarity | plementarity | plementarity | plementarity |
| mis- | and or | and or | and or | and or |
| matches | identity | identity | identity | identity |
| 0 | 100 | 100 | 100 | 100 |
| 1 | 93 | 95 | 96 | 93 |
| 2 | 87 | 90 | 92 | 87 |
| 3 | 80 | 85 | 88 | 80 |
| 4 | 73 | 80 | 84 | 73 |
| 5 | 67 | 75 | 80 | 67 |
| 6 | 60 | 70 | 76 | 60 |
| 7 | 53 | 65 | 72 | 53 |
| 8 | 47 | 60 | 68 | 47 |

It is contemplated that any embodiment discussed in this specification can be implemented with respect to any method, kit, reagent, or composition of the invention, and vice versa. Furthermore, compositions of the invention can be used to achieve methods of the invention.

In an embodiment, in instances in which each composition is administered, without limitation, as individual or separate dosage forms (e.g., capsules or tablets), the kit comprises, without limitation, the composition making up the composition of the invention, along with instructions for use. In an additional embodiment, the composition, without limitation, may be packaged in any manner suitable for administration, so long as the packaging, when considered along with the instructions for administration, without limitation, clearly indicates the manner in which the composition is to be administered. In a further embodiment, the composition or a combination of the composition with other therapeutics may, without limitation, be combined into a single administrable dosage form such as a capsule, tablet, or other solid or liquid formulation. The cancer therapeutic can be provided to an individual in a package. The package can be a container, for instance, without limitation, a bottle, a canister, a tube or other enclosed vessel. The package can also be a packet, such as a blister pack.

In an embodiment, in instances in which the composition is administered, without limitation, as individual or separate dosage forms (e.g., capsules or tablets), the kit comprises, without limitation, each of the ASO and/or SM-ASO making up the composition of the invention, along with instructions for use. In an additional embodiment, a composition, may be packaged in any manner suitable for administration, so long as the packaging, when considered along with the instructions for administration, without limitation, clearly indicates the manner in which the composition is to be administered. In a further embodiment, a composition may, without limitation, be combined into a single administrable dosage form such as a capsule, tablet, or other solid or liquid formulation. A composition can be provided to an individual in a package. The package can be a container, for instance, without limitation, a bottle, a canister, a tube or other enclosed vessel. The package can also be a packet, such as a blister pack.

In an embodiment, an individual is provided a treatment protocol wherein a composition is administered on a periodic schedule, wherein the individual is informed by electronic notification to administer the composition so that the individual is reminded to take the composition on a period schedule. In an aspect of this embodiment, the electronic notification is by email, text, instant messaging or by another electronic notification method. In an embodiment, an individual is informed to administer the composition on a period schedule through receipt of a telephone call, postal mail, overnight express (including, without limitation, FedEx and UPS) or other method of notification.

### EXAMPLES

### Example 1: Targeted ASO Screen Identified Functional IL7R ASOs

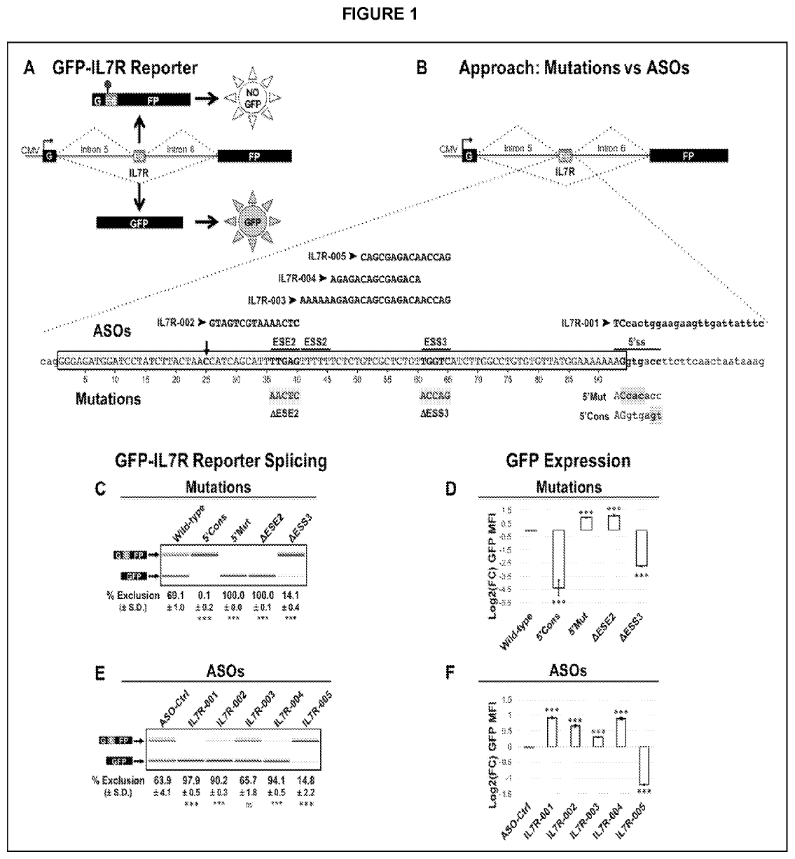

To identify ASOs that increase exclusion (also can be referred to as skipping) of IL7R exon 6, a GFP-IL7R splicing reporter was constructed. In this GFP-IL7R splicing reporter, GFP is expressed by exclusion of IL7R exon 6 (FIG. 1-A). Functional ASOs were identified by comparing the effects of ASOs blocking cis-acting splicing elements in IL7R exon 6 to the effects of mutating the corresponding elements (FIG. 1-B). Consensus (5′Cons) or crippling (5′Mut) mutations to the 5′-splice site (5′ss) force complete inclusion or exclusion of exon 6, respectively (FIG. 1-C), and set the range of GFP-IL7R splicing report expression (FIG. 1-D). We designed five ASOs (IL7R-001—IL7R-005) targeting different cis-acting elements in exon 6. ASO IL7R-001 was designed to block the 5′ss of exon 6, and ASO IL7R-002 was designed to block the exonic splicing enhancer 2 (ESE2) in exon 6 (FIG. 1-B). Morpholino ASOs of the desired sequence were synthesized by Gene Tools. The ASOs were transfected into a HeLa cell line that stably expressed the GFP-IL7R reporter. The transfection was accomplished using the Endo-Porter transfection reagent (Gene Tools, Cat #SKU: OT-EP-PEG-1) and cells were assayed for IL7R exon 6 splicing (RT-PCR) and GFP expression using flow cytometry (Guava easyCyte, Luminex Corp). Using these methods, we uncovered three ASOs (IL7R-001, IL7R-002 and IL7R-004) that enhanced exon 6 exclusion (FIG. 1-E) and GFP expression (FIG. 1-F). IL7R-001 and IL7R-002 enhanced exon 6 exclusion in a manner similar to the mutation of their corresponding cis-acting element (5′Mut and AESE2, respectively), thereby confirming that each was able to splice modulate. We found that ASOs IL7R-001 and IL7R-004 had the greatest potency to enhance exon 6. These two ASOs were selected as lead ASOs for further development for use as part of an immuno-oncology treatment. These two ASOs are hereafter collectively referred to as pro-sIL7R ASOs. We also identified additional ASOs, namely, IL7R-005 and IL7R-006 (not shown) that reduced exon 6 exclusion. These ASO are under development to treat autoimmune disorders.

### Example 2: ASO-Walk Screen Targeting the Flanking Introns Identified Additional Functional ASOs

We conducted an ASO-Walk screen with ASOs targeting IL7R introns 5 and 6 using the GFP-IL7R reporter cell line generated in Example 1. This ASO walk screen is similar to the approach undertaken in the discovery of Spinraza as set forth in Hua (Hua, Y., Vickers, T. A., Baker, B. F., Bennett, C. F., and Krainer, A. R. (2007). Enhancement of SMN2 exon 7 inclusion by antisense oligonucleotides targeting the exon. PLoS Biol 5, e73).

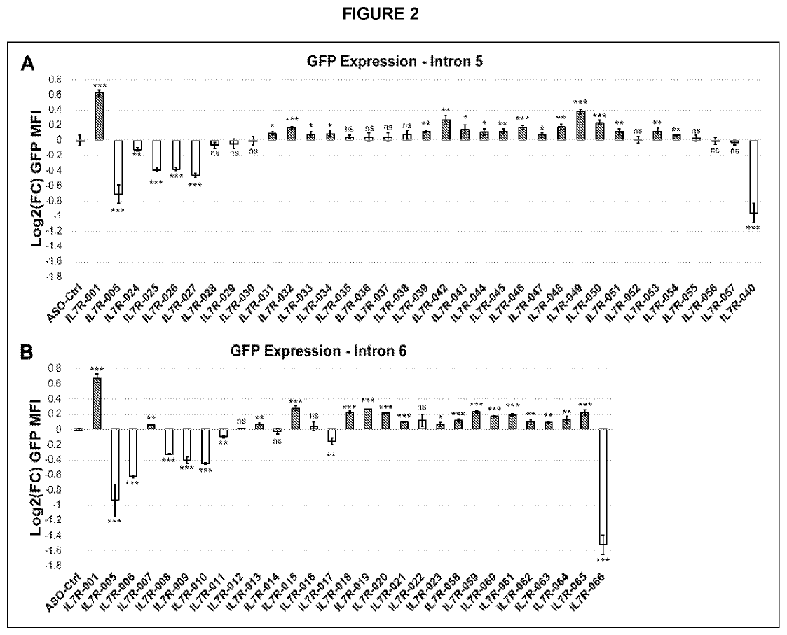

The morpholino ASOs used were 15-25 nt in length and targeted overlapping sequences starting in the intronic regions closest to exon 6 and moving away from the exon in 5 nt stretches. The ASOs were transfected into the GFP-IL7R reporter cell line with Endo-Porter transfection reagent (Gene Tools, Cat #SKU: OT-EP-PEG-1) and cells were assayed for GFP expression as disclosed in Example 1. We screened a total of 57 ASOs, of which 32 targeted intron 5 (FIG. 2-A) and 25 targeted intron 6 (FIG. 2-B). In all experiments, ASO IL7R-001 was used as positive control. In addition to ASOs IL7R-001, IL7R-002, IL7R-003 and IL7R-004, this ASO-Walk approach uncovered an additional 33 ASOs that increased GFP expression in a statistically significant manner (highlighted in yellow in FIGS. 2-A & 2-B; Tables 3 & 4). IL7R-001 or IL7R-004 were found to be the most potent ASOs and were selected as lead pro-sIL7R ASOs for further development.

### Example 3: Lead ASOs Modulate IL7R Splicing in a Concentration-Dependent Fashion

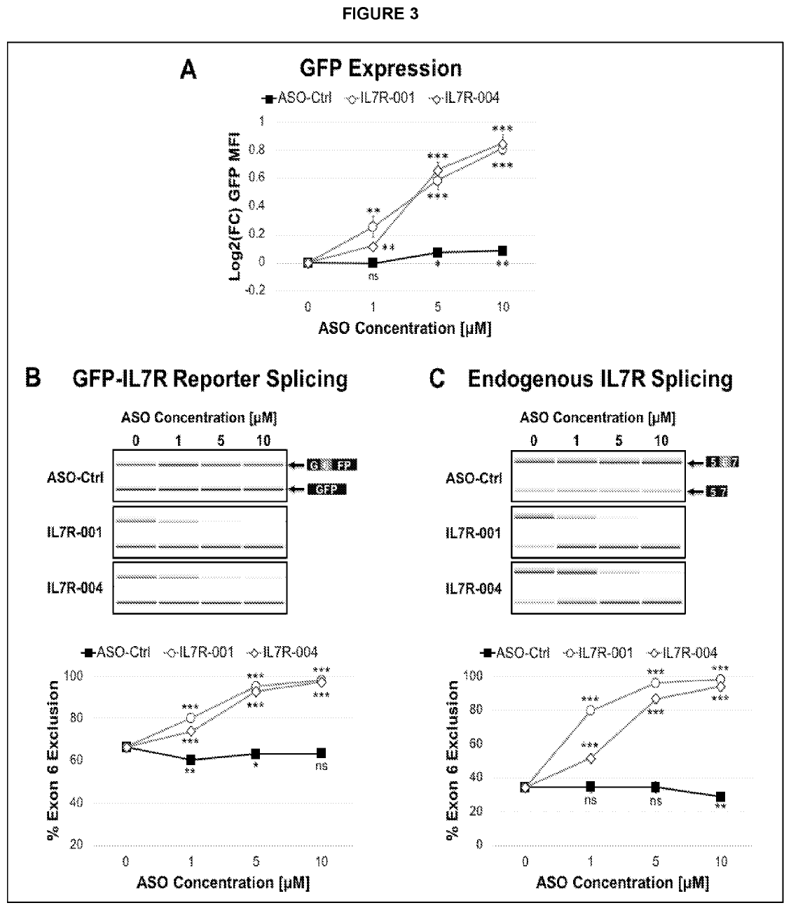

Dose-response studies were conducted with IL7R-001 or IL7R-004 to determine whether exon 6 exclusion could be dose dependent based on concentration of the ASO. To this end IL7R-001 or IL7R-004 morpholino ASOs were transfected into the GFP-IL7R reporter cell line at various concentrations. We determined that increasing concentrations (0, 1 M, 5 M and 10 M) of ASOs IL7R-001 and IL7R-004 progressively increased GFP expression (FIG. 3-A) and exon 6 exclusion (FIG. 3-B). In contrast, increasing concentrations of the control ASO (ASO-Ctrl; Gene Tools, cat #SKU: PCO-StandardControl-100) did not. We found that this concentration-dependent increase in exon 6 exclusion was replicated in transcripts from the endogenous IL7R gene (FIG. 3-C).

### Example 4: Lead ASOs Modulate sIL7R Secretion in HeLa Cells

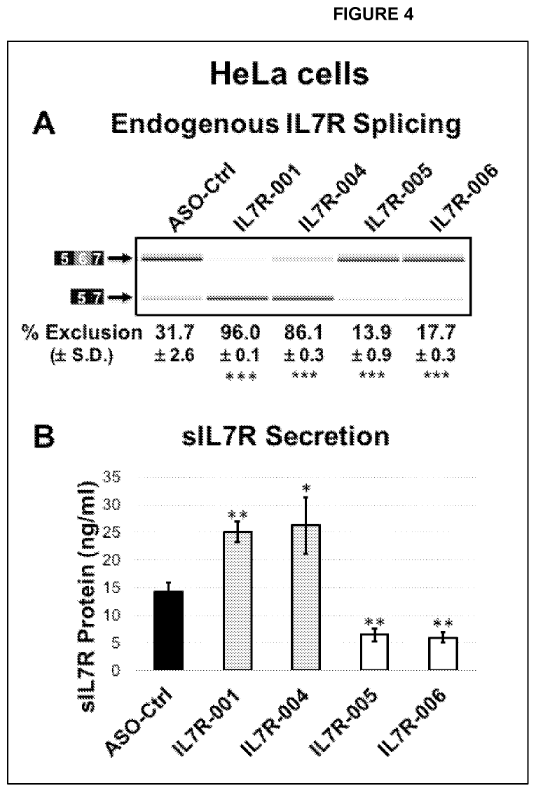

We next assessed the effect of ASOs IL7R-001 and IL7R-004 on the secretion of sIL7R. Control (ASO-Ctrl) and experimental (IL7R-001, IL7R-004, IL7R-005 and IL7R-006) morpholino ASOs were transfected into HeLa cells at 10 μM with the Endo-Porter transfection reagent as in Example 1. Three days later, cells were assayed for exon 6 splicing and sIL7R secretion. RT-PCR analysis of transfected cells found 31.7% exon 6 exclusion in cells transfected with the ASO-Ctrl. When the cells were transfected with ASOs IL7R-001 and IL7R-004, exon 6 exclusion was 96.0% and 86.1%, respectively (FIG. 4-A). Exon 6 exclusion was reduced by ASOs IL7R-005 (13.9%) and IL7R-006 (17.7%) (FIG. 4-A). ASOs IL7R-001 and IL7R-004 increased sIL7R secretion by 1.8-fold and 1.9-fold, respectively, and ASOs IL7R-005 and IL7R-006 decreased sIL7R secretion by 2.2-fold and 2.4-fold, respectively (FIG. 4-B). These results showed that ASOs IL7R-001 and IL7R-004 were able to modulate the expression of sIL7R when transfected into HeLa cells in direct proportion to their effect on exon 6 exclusion.

### Example 5: Lead ASOs Modulate sIL7R Secretion in Human Primary CD4⁺ T Cells with Minimal Impact on mIL7R Expression

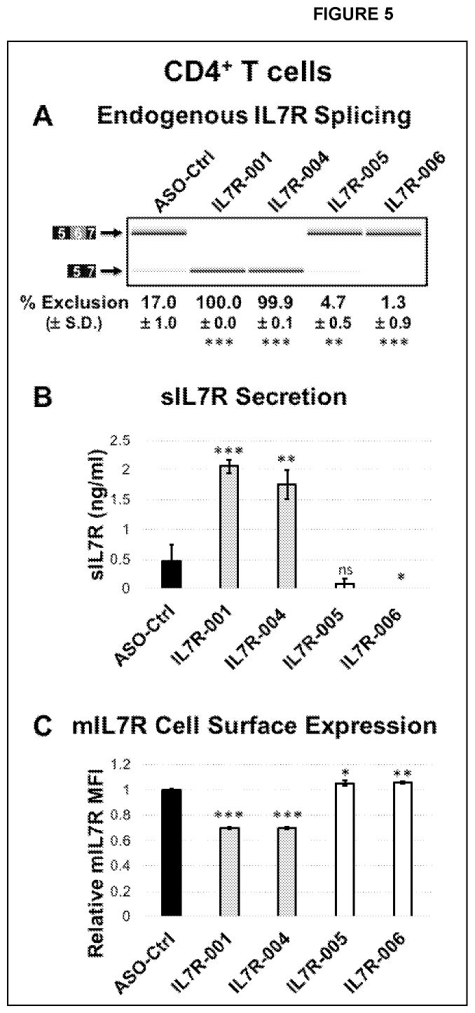

Given that IL7R is predominantly expressed in T cells, this cell type is the major source of sIL7R production in vivo. Therefore, we next evaluated the effect of ASOs IL7R-001 and IL7R-004 on the secretion of sIL7R in human primary CD4⁺ T cells isolated from healthy human donors. ASOs IL7R-001 and IL7R-004 were transfected into the human primary CD4⁺ T cells by nucleofection (Amaxa Nucleofector, Lonza) and the cells were assayed three days after to identify exon 6 splicing and sIL7R secretion. While exon 6 was excluded in 17.0% of IL7R transcripts in T cells nucleofected with the ASO-Ctrl, it was completely excluded with ASOs IL7R-001 (100.0%) and IL7R-004 (99.9%). In contrast, exclusion was essentially abolished with ASOs IL7R-005 (4.7%) and IL7R-006 (1.3%) (FIG. 5-A). This modulation of exon 6 splicing by ASOs IL7R-001 and IL7R-004 led to changes in the secretion of sIL7R that were expected. ASOs IL7R-001 and IL7R-004 significantly increased sIL7R secretion (4.5-fold and 3.8-fold, respectively), whereas ASOs IL7R-005 and IL7R-006 decreased sIL7R secretion by greater than 5-fold (reduced to near or below the limit of detection) (FIG. 5-B). Since ASOs IL7R-001 and IL7R-004 enhance sIL7R secretion, these are hereafter collectively referred to as pro-sIL7R ASOs. ASOs IL7R-005 and IL7R-006 reduce sIL7R secretion, and thus are hereafter collectively referred to as anti-sIL7R ASOs. Based on these results, these IL7R ASOs have potential therapeutic utility in autoimmunity (anti-sIL7R ASOs) and cancer (pro-sIL7R ASOs). These results show that pro-sIL7R ASOs, IL7R-001 and IL7R-004 effectively increased sIL7R expression in human CD4⁺ T cells, thereby satisfying an efficacy endpoint for a cancer immunotherapy therapeutic.

Expression of mIL7R on the surface of human primary CD4⁺ T cells plays a central role in the development and survival of T cells (Fry, T. J., and Mackall, C. L. (2005). The many faces of IL-7: from lymphopoiesis to peripheral T cell maintenance. Journal of immunology 174, 6571-6576; Mazzucchelli, R., and Durum, S. K. (2007). Interleukin-7 receptor expression: intelligent design. Nat Rev Immunol 7, 144-154), as evidenced by lymphopenia and severe immunodeficiency observed in IL7R knockout in mice and loss-of-function mutations in humans (Maraskovsky, E., Teepe, M., Morrissey, P. J., Braddy, S., Miller, R. E., Lynch, D. H., and Peschon, J. J. (1996). Impaired survival and proliferation in IL-7 receptor-deficient peripheral T cells. Journal of immunology 157, 5315-5323; Peschon, J. J., Morrissey, P. J., Grabstein, K. H., Ramsdell, F. J., Maraskovsky, E., Gliniak, B. C., Park, L. S., Ziegler, S. F., Williams, D. E., Ware, C. B., et al. (1994). Early lymphocyte expansion is severely impaired in interleukin 7 receptor-deficient mice. J Exp Med 180,1955-1960; Puel, A., Ziegler, S. F., Buckley, R. H., and Leonard, W. J. (1998). Defective IL7R expression in T(−)B(+)NK(+) severe combined immunodeficiency. Nature genetics 20, 394-397; Roifman, C. M., Zhang, J., Chitayat, D., and Sharfe, N. (2000). A partial deficiency of interleukin-7R alpha is sufficient to abrogate T-cell development and cause severe combined immunodeficiency. Blood 96, 2803-2807). Accordingly, therapies that severely inhibit the expression and/or function of mIL7R are predicted to cause broad immunosuppression that could put patients at risk of serious infections and cancers. This is not a concern for anti-sIL7R ASOs since these are predicted to decrease sIL7R expression without reducing mIL7R expression. Indeed, anti-sIL7R ASOs IL7R-005 and IL7R-006 did not reduce mIL7R cell surface expression in primary CD4⁺ T cells (FIG. 5-C), and thus these ASOs avoid immunosuppression. ASOs IL7R-005 and IL7R-006 are excellent candidates to provide safer treatment for Multiple Sclerosis and autoimmunity by reducing side effects associated with immunosuppression.

Cell surface expression of mIL7R in the primary CD4⁺ T cells treated with pro-sIL7R ASOs, IL7R-001 and IL7R-004 was reduced ˜30% (1.4-fold) compared to T cells treated with control ASO (FIG. 5-C). This reduction is tolerable since heterozygous knockout of IL7R in mice is tolerated.

### Example 6: Pro-sIL7R ASOs Phenocopy Mis-Splicing of Exon 6 Caused by the MS-Associated SNP rs6897932 with Potential Applications in Immuno-Oncology

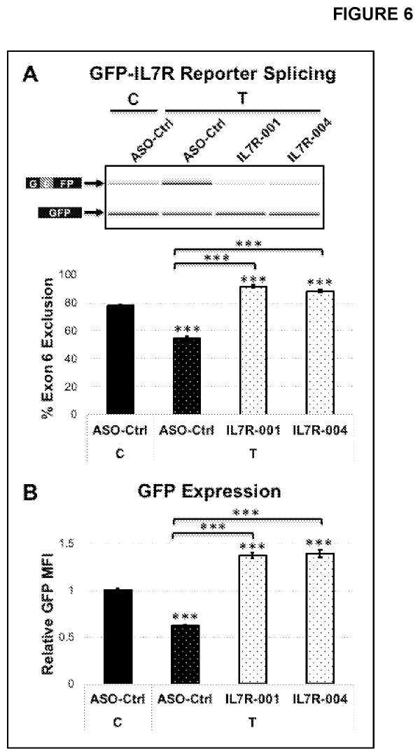

In this example we test whether ASOs IL7R-001 and IL7R-004 can increase sIL7R expression to drive T cell-derived self-reactivity to enhance anti-cancer immunity. To carry out this test, we started with the knowledge that the SNP rs6897932 is a key determinant of sIL7R expression, that sIL7R is an activator of T cell activity, in particular self-reactive T cells, and that immune reactions against cancer cells are self-reactive in nature. Thus, to test if ASOs IL7R-001 an IL7R-004 can be used to increase anti-cancer immunity, we started with the premise that sIL7R needs to be raised to or above the levels observed in individuals homozygous for the risk ‘C’ allele at rs6897932, as these individuals are predisposed to self-reactivity. To carry out this test, we used pro-sIL7R ASOs IL7R-001 and IL7R-004, and versions of the GFP-IL7R reporter carrying either the risk ‘C’ allele (C reporter) or the protective ‘T’ allele (T reporter) of rs6897932. For comparison, we stably transfected the ASO-Ctrl into the HeLa cell lines to express either the C reporter or the T reporter; creating conditions that mimic the levels of exon 6 exclusion in individuals with high propensity (C reporter) or low propensity (T reporter) for self-reactivity. Illustrating the effects of the SNP (C versus T), RT-PCR analysis in cells treated with ASO-Ctrl found that exon 6 was excluded at a significantly higher frequency (78.0%) in the C reporter cell line than in the T reporter cell line (54.4%) (FIG. 6-A). Finally, we transfected the HeLa cell line to stably express the T reporter with IL7R-001 or IL7R-004 to see if these ASOs can enhance exon 6 exclusion to levels similar to those observed with the C reporter cell line transfected with ASO-Ctrl. The results show that pro-sIL7R ASOs IL7R-001 and IL7R-004 enhanced exon 6 exclusion in the T reporter cell line to levels greater than those observed with the C reporter cell line, reaching 91.5% and 88.3% exclusion, respectively (FIG. 6-A). Because GFP expression from the reporter is a proxy for sIL7R, we also examined effects of the ASOs on GFP expression. GFP expression in the T reporter cells treated with IL7R-001 and IL7R-004 was significantly higher (˜1.4 fold) than in the C reporter cells treated with ASO-Ctrl (FIG. 6-B). These results found that pro-sIL7R ASOs IL7R-001 and IL7R-004 can enhance exclusion of IL7R exon 6 to levels above those observed in individuals with high predisposition to self-reactivity. It is contended that pro-sIL7R ASOs IL7R-001 and IL7R-004 would make good candidates to boost anti-cancer immunity.

### Example 7: Ex Vivo Delivery to Target Cells and the Development of ASO IL7R-001

We next examined the ability of pro-sIL7R ASOs to modify sIL7R expression in human primary T cells ex vivo without the need of a transfection method. This is important because delivery of ASOs into T cells without transfection is a difficult task. For this purpose, we screened the ASO IL7R-001 that had been modified through different chemical modifications or conjugated to a moiety that could improve its delivery and activity into human primary T cells ex vivo. To this end, the modified or conjugated ASOs, ASO-Ctrl or IL7R-001 were added to the media to determine if the ASOs were able to enter the T cells without the use of a transfection method (unassisted entry into the cells). Through this work, we identified a delivery moiety that, when conjugated to the morpholino ASO IL7R-001, was able to greatly enhance exclusion of IL7R exon 6 in human primary T cells, while the conjugated ASO-Ctrl did not (data not shown). Thus, it was clear that the conjugated ASO IL7R-001 efficiently entered into T cells ex vivo without the need of transfection. Thus, we showed that we could get the ASOs to enter the T cells by natural cellular mechanisms that do not require transfection, which is referred to as gymnotic delivery.

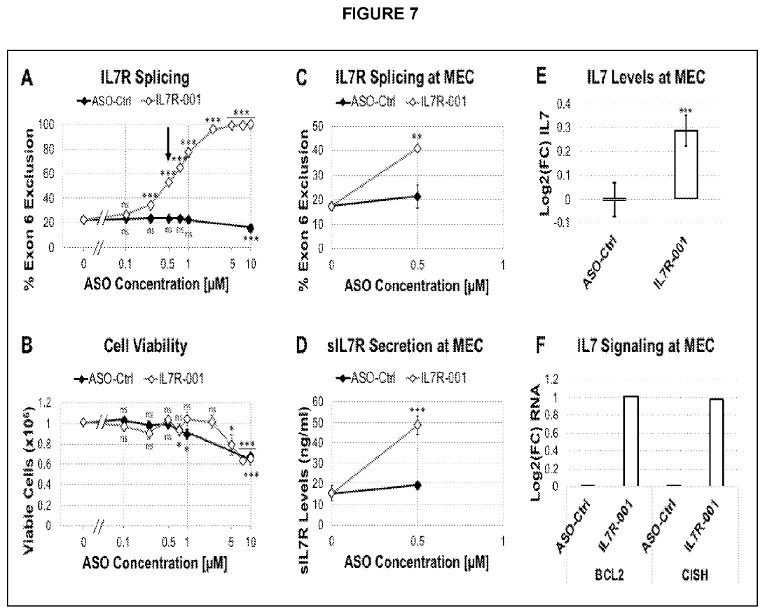

We selected ASO IL7R-001 with the conjugated delivery moiety for use in further functional studies by gymnotic delivery in human primary CD4⁺ T cells ex vivo. The first study we conducted was a dose-response study to determine the minimum effective concentration (MEC) of ASO IL7R-001. These studies were conducted by gymnotic delivery in human primary CD4⁺ T cells followed by assessment of the efficacy of these cells to modulate exon 6 splicing. We also examined potential cellular toxicity. We were looking to see which was the concentration at which we could increase exon 6 exclusion to 50% (FIG. 7-A, black arrow) without causing adverse toxic effects to the cells (FIG. 7-B). We found this minimum effective concentration (MEC) of IL7R-001 to be 0.5 μM. We next validated that IL7R-001 at the MEC effectively increased exon 6 exclusion (FIG. 7-C) and sIL7R secretion by 2.5-fold (FIG. 7-D) with minimal reduction in mIL7R cell surface expression (data not shown). These results mimic the effects of the Multiple Sclerosis risk SNP rs6897932 in IL7R, which drives self-reactivity in Multiple Sclerosis by enhancing exon 6 exclusion and sIL7R expression by ˜3-fold.

Next, we investigated the effects of enhancing sIL7R levels on T cell function by ASO IL7R-001. This study examined the functional effects of IL7R-001 on IL7 signaling in T cells, since sIL7R has been shown to drive self-reactivity by potentiating IL7 availability and activity (Lundstrom, W., Highfill, S., Walsh, S. T., Beq, S., Morse, E., Kockum, I., Alfredsson, L., Olsson, T., Hillert, J., and Mackall, C. L. (2013). Soluble IL7Ralpha potentiates IL-7 bioactivity and promotes autoimmunity. Proceedings of the National Academy of Sciences of the United States of America 110, E1761-1770). Compared to the control ASO (ASO-Ctrl), we found that increased sIL7R secretion caused by administration of ASO IL7R-001 resulted in an enhancement of the availability of IL7 (FIG. 7-E) leading to increased expression of the IL7-induced genes BCL2 and CISH (FIG. 7-F). These results showed that ASO IL7R-001 potentiates IL7 signaling by enhancing sIL7R.

### Example 8: Effects of IL7R-001 on T Cell Function

We examined the duration of the potentiation of IL7 by ASO IL7R-001. This was done by measuring BCL2 expression five days after IL7 treatment. The results showed that enhanced BCL2 expression by ASO IL7R-001 is maintained five days after treatment (FIG. 8-A). This shows a lasting potentiation of IL7 by ASO IL7R-001. We further showed that the elevated levels of sIL7R over the five-day period induced by ASO IL7R-001 treatment (FIG. 8-B), led to the enhanced survival of human primary CD4⁺ T cells ex vivo (FIG. 8-C). These results support the potential of ASO IL7R-001 to enhance T cell activity for cancer treatment.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, “without limitation”, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature.

In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, or AIA 35 U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

1.-11. (canceled)

12. A composition comprising an oligonucleotide that binds to a sequence in pre-mRNAs of an interleukin 7 receptor (IL7R) that influences splicing of exon 6,
wherein the oligonucleotide increases exclusion of exon 6 in IL7R pre-mRNAs and increases expression of the soluble isoform of IL7R (sIL7R).

13. The composition of claim 12, wherein the oligonucleotide is an antisense oligonucleotide (ASO) or a splice-modulating antisense oligonucleotide (SM-ASO).

14. The composition of claim 12, wherein the oligonucleotide is selected from SEQ ID No. 27 or SEQ ID No. 30, or a sequence having at least 70, 75, 80, 84, 85, 88, 92, 93, 94, 95, or 96% complementarity to SEQ ID No. 64 or SEQ ID No. 67.

15. The composition of claim 12, wherein the composition is adapted for administration to treat a cancer.

16. The composition of claim 12, wherein the composition further comprises a pharmaceutically acceptable excipient, salts, or carrier.

17. (canceled)

18. The composition of claim 12, wherein the composition further comprises one or more active agents

19. The composition of claim 12, wherein one or more of (1) the ribose or other sugar units, (2) the bases, or (3) the backbone of the oligonucleotide are modified.

20. The composition of claim 12, wherein the composition is modified with nucleotides with phosphate modifications comprising one or more phosphorothioate, phosphorodithioate, phosphodiester, methyl phosphonate, phosphoramidate, methylphosphonate, phosphotriester, phosphoroaridate, morpholino, amidate carbamate, carboxymethyl, acetamidate, polyamide, sulfonate, sulfonamide, sulfamate, formacetal, thioformacetal, or alkylsilyl substitutions and combinations of two or more of any of the foregoing.

21. The composition of claim 12, wherein the composition is modified by a sugar modifications such as 2′-O-methyl (2′-O-methylnucleotides) and 2′-O-methyloxyethoxy (2′-O-MOE), a 2′-O-alkyl modified sugar moiety, or a bicyclic sugar moiety, and nucleotide mimetics such as, without limitation, peptide nucleic acids (PNA), morpholino nucleic acids, cyclohexenyl nucleic acids, anhydrohexitol nucleic acids, glycol nucleic acid, threose nucleic acid, and locked nucleic acids (LNA), as well as partially or completely modified backbones, such as fully modified sugar phosphate backbone, a locked nucleic acid backbone, a peptidic backbone, a phosphotriester backbone, a phosphoramidate backbone, a siloxane backbone, a carboxymethylester backbone, an acetamidate backbone, a carbamate backbone, a thioether backbone, a bridged methylene phosphonate backbone, a phosphorothioate backbone, a methylphosphonate backbone, an alkylphosphonate backbone, a phosphate ester backbone, an alkylphosphonothioate backbone, a phosphorodithioate backbone, a carbonate backbone, a phosphate triester backbone, a carboxymethyl ester backbone, a methylphosphorothioate backbone, a phosphorodithioate backbone, a backbone having p-ethoxy linkages, and combinations of two or more of any of the foregoing.

22.-108. (canceled)

109. The composition of claim 12, wherein the oligonucleotide is modified by a peptide, a cell penetrating peptide, an antibody, a nanobody, a camelid, an antibody variable region, a small molecule, and/or a ligand that enhances the stability, distribution or delivery of the oligonucleotide to specific tissues.

110. The composition of claim 18, wherein the one or more active agents for treating cancer are selected from immune check point inhibitors, therapeutic antibodies, chemotherapy agents and therapeutic radiation.

111. The composition of claim 12, wherein the oligonucleotide has a sequence with at least 70%, at least 75%, at least 80%, at least 85%, at least 90%, at least 95% or 100% identity to a nucleotide selected from SEQ ID NOS: 27-63.

112. The composition of claim 12, wherein the oligonucleotide binds fully or partially to a sequence with at least 70%, at least 75%, at least 80%, at least 85%, at least 90%, at least 95% or 100% identity to a nucleotide selected from SEQ ID NOS: 64-100.

113. A method of treating cancer, the method comprising administering a therapeutic amount of the composition of claim 12 to a subject.

114. The method of claim 113, wherein the cancer is one or more of colorectal cancer, liver cancer, cholangiocarcinoma, hepatocellular carcinoma, melanoma, lung cancer, adenocarcinoma, squamous cell carcinoma, large cell (undifferentiated) carcinoma, non-small cell lung cancer (NSCLC), small cell lung cancer, kidney cancer, renal cell carcinoma, squamous cell carcinoma of the esophagus, head and neck cancer, urothelial carcinoma, cervical cancer, cutaneous squamous cell carcinoma, endometrial carcinoma, gastric cancer, gastroesophageal junction cancer, esophageal cancer, microsatellite instability-high (MSI-H) or mismatch repair deficient (dMMR) cancer, solid tumors, triple-negative breast cancer, triple-positive breast cancer, brain cancer, astrocytoma, ependymoma, glioma, meningioma, medulloblastoma, neuroblastoma, bladder cancer, childhood cancer, multiple myeloma, ovarian cancer, pancreatic cancer, prostate cancer, sarcoma, skin cancer, melanoma, Merkel cell carcinoma (MCC), Kaposi's sarcoma (KS), Stomach cancer and uterine cancer.

115. The method of claim 113, wherein the composition is administered with a second therapeutic agent, wherein the second therapeutic agent selected from an immune check point inhibitor, a therapeutic antibody, a chemotherapy agent or therapeutic radiation.

116. The method of claim 113, wherein the composition enhances the activity or response rate of an immunotherapy.

117. The method of claim 116, wherein the immunotherapy comprises administration of an immune-checkpoint inhibitor.

118. A method of enhancing a response to a cancer treatment, the method comprising administering a therapeutic amount of the composition of claim 12 to a subject.

119. The method of claim 118, wherein the cancer treatment comprises administration of an immune-checkpoint inhibitor.


SEQUENCE LISTING (100 sequences)

SEQ ID NO: 1 - DNA, 15 bp, artificial
  IL7R-005
gaccaacagagcgac

SEQ ID NO: 2 - DNA, 20 bp, artificial
  IL7R-006
cacaaucacccucuuuauua

SEQ ID NO: 3 - DNA, 15 bp, artificial
  IL7R-008 (Cluster 2)
cgugaucccacacaa

SEQ ID NO: 4 - DNA, 15 bp, artificial
  IL7R-009 (Cluster 2)
cuguccgugauccca

SEQ ID NO: 5 - DNA, 15 bp, artificial
  IL7R-010 (Cluster 2)
ucugacuguccguga

SEQ ID NO: 6 - DNA, 25 bp, artificial
  IL7R-066 (Entire sequence of Cluster 2)
ucugacuguccgugaucccacacaa

SEQ ID NO: 7 - DNA, 18 bp, artificial
  IL7R-011
gcuuaagcucugacuguc

SEQ ID NO: 8 - DNA, 15 bp, artificial
  IL7R-017
uuugugguuuucuca

SEQ ID NO: 9 - DNA, 15 bp, artificial
  IL7R-024
caugcaguggaggua

SEQ ID NO: 10 - DNA, 15 bp, artificial
  IL7R-025 (Cluster 1)
aguggagguaggguc

SEQ ID NO: 11 - DNA, 15 bp, artificial
  IL7R-026 (Cluster 1)
ggguagggucucagg

SEQ ID NO: 12 - DNA, 15 bp, artificial
  IL7R-027 (Cluster 1)
gggucucagggugcu

SEQ ID NO: 13 - DNA, 25 bp, artificial
  IL7R-041 (Entire sequence of Cluster 1)
aguggugguagggucucagggugcu

SEQ ID NO: 14 - DNA, 25 bp, artificial
  IL7R-001
cuuuauuaguugaagaaggucaccu

SEQ ID NO: 15 - DNA, 15 bp, artificial
  IL7R-002
cucaaaaugcugaug

SEQ ID NO: 16 - DNA, 25 bp, artificial
  IL7R-003
gaccaacagagcgacagagaaaaaa

SEQ ID NO: 17 - DNA, 15 bp, artificial
  IL7R-004
acagagcgacagaga

SEQ ID NO: 18 - DNA, 15 bp, artificial
  IL7R-007
ucccacacaaucacc

SEQ ID NO: 19 - DNA, 22 bp, artificial
  IL7R-013
caucaauaaaugggacuuaagc

SEQ ID NO: 20 - DNA, 21 bp, artificial
  IL7R-015
ugguuuucucaucaauaaaug

SEQ ID NO: 21 - DNA, 15 bp, artificial
  IL7R-018
uccccuuugugguuu

SEQ ID NO: 22 - DNA, 16 bp, artificial
  IL7R-019
ccuuaauccccuuugu

SEQ ID NO: 23 - DNA, 18 bp, artificial
  IL7R-020
ugaaaugccuuaaucccc

SEQ ID NO: 24 - DNA, 16 bp, artificial
  IL7R-021
ucgugaaaugccuuaa

SEQ ID NO: 25 - DNA, 16 bp, artificial
  IL7R-023
ggcacuaaauucguga

SEQ ID NO: 26 - DNA, 18 bp, artificial
  IL7R-031
uuuacuuaguaauguggg

SEQ ID NO: 27 - DNA, 15 bp, artificial
  IL7R-032
uuaguaaugugggcc

SEQ ID NO: 28 - DNA, 15 bp, artificial
  IL7R-033
aauguggacccacuu

SEQ ID NO: 29 - DNA, 15 bp, artificial
  IL7R-034
ggguccacuuauuau

SEQ ID NO: 30 - DNA, 19 bp, artificial
  IL7R-039
uugcuuuucaguuaagaga

SEQ ID NO: 31 - DNA, 22 bp, artificial
  IL7R-042
uuucaguuaagagacauauuug

SEQ ID NO: 32 - DNA, 20 bp, artificial
  IL7R-043
guuaagagacauauuugaca

SEQ ID NO: 33 - DNA, 17 bp, artificial
  IL7R-044
gagacauauuugacagc

SEQ ID NO: 34 - DNA, 17 bp, artificial
  IL7R-045
auauuugacagcuuuau

SEQ ID NO: 35 - DNA, 15 bp, artificial
  IL7R-046
ugacagcuuuaugga

SEQ ID NO: 36 - DNA, 15 bp, artificial
  IL7R-047
gcuuuacggagggau

SEQ ID NO: 37 - DNA, 17 bp, artificial
  IL7R-048
auggagggauuuugguu

SEQ ID NO: 38 - DNA, 21 bp, artificial
  IL7R-049
gggauuuugguuuaaaaggca

SEQ ID NO: 39 - DNA, 21 bp, artificial
  IL7R-050
uuugguuuaaaaggcauugac

SEQ ID NO: 40 - DNA, 21 bp, artificial
  IL7R-051
uuuaaaaggcauugacuuggg

SEQ ID NO: 41 - DNA, 15 bp, artificial
  IL7R-053
auugacuugggugac

SEQ ID NO: 42 - DNA, 15 bp, artificial
  IL7R-054
cuugggugaccaggc

SEQ ID NO: 43 - DNA, 15 bp, artificial
  IL7R-058
acugggcacuaaauu

SEQ ID NO: 44 - DNA, 15 bp, artificial
  IL7R-059
gggauacugggcacu

SEQ ID NO: 45 - DNA, 15 bp, artificial
  IL7R-060
agauagggauacugg

SEQ ID NO: 46 - DNA, 17 bp, artificial
  IL7R-061
ugaggauagauagggau

SEQ ID NO: 47 - DNA, 15 bp, artificial
  IL7R-062
cgcugaggauagaua

SEQ ID NO: 48 - DNA, 16 bp, artificial
  IL7R-063
gaaauucgcugaggau

SEQ ID NO: 49 - DNA, 15 bp, artificial
  IL7R-064
uguggaaauucgcug

SEQ ID NO: 50 - DNA, 24 bp, artificial
  IL7R-065
cuuaugaaauuaacuguggaaauu

SEQ ID NO: 51 - DNA, 15 bp, Homo sapien
gucgcucuguugguc

SEQ ID NO: 52 - DNA, 20 bp, Homo sapien
uaauaaagagggugauugug

SEQ ID NO: 53 - DNA, 15 bp, Homo sapien
uugugugggaucacg

SEQ ID NO: 54 - DNA, 15 bp, Homo sapien
ugggaucacggacag

SEQ ID NO: 55 - DNA, 15 bp, Homo sapien
ucacggacagucaga

SEQ ID NO: 56 - DNA, 25 bp, Homo sapien
uugugugggaucacggacagucaga

SEQ ID NO: 57 - DNA, 18 bp, Homo sapien
gacagucagagcuuaagc

SEQ ID NO: 58 - DNA, 15 bp, Homo sapien
ugagaaaaccacaaa

SEQ ID NO: 59 - DNA, 15 bp, Homo sapien
uacccccacugcaug

SEQ ID NO: 60 - DNA, 15 bp, Homo sapien
gacccuacccccacu

SEQ ID NO: 61 - DNA, 15 bp, Homo sapien
ccugagacccuaccc

SEQ ID NO: 62 - DNA, 15 bp, Homo sapien
agcacccugagaccc

SEQ ID NO: 63 - DNA, 25 bp, Homo sapien
agcacccugagacccuacccccacu

SEQ ID NO: 64 - DNA, 25 bp, Homo sapien
aggugaccuucuucaacuaauaaag

SEQ ID NO: 65 - DNA, 15 bp, Homo sapien
caucagcauuuugag

SEQ ID NO: 66 - DNA, 25 bp, Homo sapien
uuuuuucucugucgcucuguugguc

SEQ ID NO: 67 - DNA, 15 bp, Homo sapien
ucucugucgcucugu

SEQ ID NO: 68 - DNA, 15 bp, Homo sapien
ggugauuguguggga

SEQ ID NO: 69 - DNA, 22 bp, Homo sapien
gcuuaagccccauuuauugaug

SEQ ID NO: 70 - DNA, 21 bp, Homo sapien
cauuuauugaugagaaaacca

SEQ ID NO: 71 - DNA, 15 bp, Homo sapien
aaaccacaaagggga

SEQ ID NO: 72 - DNA, 16 bp, Homo sapien
acaaaggggauuaagg

SEQ ID NO: 73 - DNA, 18 bp, Homo sapien
ggggauuaaggcauuuca

SEQ ID NO: 74 - DNA, 16 bp, Homo sapien
uuaaggcauuucacga

SEQ ID NO: 75 - DNA, 16 bp, Homo sapien
ucacgaauuuagugcc

SEQ ID NO: 76 - DNA, 18 bp, Homo sapien
cccacauuacuaaguaaa

SEQ ID NO: 77 - DNA, 15 bp, Homo sapien
ggcccacauuacuaa

SEQ ID NO: 78 - DNA, 15 bp, Homo sapien
aagugggcccacauu

SEQ ID NO: 79 - DNA, 15 bp, Homo sapien
auaauaagugggccc

SEQ ID NO: 80 - DNA, 19 bp, Homo sapien
ucucuuaacugaaaagcaa

SEQ ID NO: 81 - DNA, 22 bp, Homo sapien
caaauaugucucuuaacugaaa

SEQ ID NO: 82 - DNA, 20 bp, Homo sapien
ugucaaauaugucucuuaac

SEQ ID NO: 83 - DNA, 17 bp, Homo sapien
gcugucaaauaugucuc

SEQ ID NO: 84 - DNA, 17 bp, Homo sapien
auaaagcugucaaauau

SEQ ID NO: 85 - DNA, 15 bp, Homo sapien
uccauaaagcuguca

SEQ ID NO: 86 - DNA, 15 bp, Homo sapien
aucccuccauaaagc

SEQ ID NO: 87 - DNA, 17 bp, Homo sapien
aaccaaaaucccuccau

SEQ ID NO: 88 - DNA, 21 bp, Homo sapien
ugccuuuuaaaccaaaauccc

SEQ ID NO: 89 - DNA, 21 bp, Homo sapien
gucaaugccuuuuaaaccaaa

SEQ ID NO: 90 - DNA, 21 bp, Homo sapien
cccaagucaaugccuuuuaaa

SEQ ID NO: 91 - DNA, 15 bp, Homo sapien
gucacccaagucaau

SEQ ID NO: 92 - DNA, 15 bp, Homo sapien
gccuggucacccaag

SEQ ID NO: 93 - DNA, 15 bp, Homo sapien
aauuuagugcccagu

SEQ ID NO: 94 - DNA, 15 bp, Homo sapien
agugcccaguauccc

SEQ ID NO: 95 - DNA, 15 bp, Homo sapien
ccaguaucccuaucu

SEQ ID NO: 96 - DNA, 17 bp, Homo sapien
aucccuaucuauccuca

SEQ ID NO: 97 - DNA, 15 bp, Homo sapien
uaucuauccucagcg

SEQ ID NO: 98 - DNA, 16 bp, Homo sapien
auccucagcgaauuuc

SEQ ID NO: 99 - DNA, 15 bp, Homo sapien
cagcgaauuuccaca

SEQ ID NO: 100 - DNA, 24 bp, Homo sapien
aauuuccacaguuaauuucauaag